(19) United States
(12) Patent Application Publication
(10) Pub. No.: US 2017/0311826 A1
(43) Pub. Date: Nov. 2, 2017

(54) SENSOR UNIT, BIOLOGICAL INFORMATION DETECTION DEVICE, ELECTRONIC APPARATUS, AND BIOLOGICAL INFORMATION DETECTION METHOD

(71) Applicant: SEIKO EPSON CORPORATION, Tokyo (JP)

(72) Inventor: Atsushi NARUSAWA, Chino-shi (JP)

(73) Assignee: SEIKO EPSON CORPORATION, Tokyo (JP)

(21) Appl. No.: 15/531,405

(22) PCT Filed: Nov. 19, 2015
(86) PCT No.: PCT/JP2015/005787

§ 371 Date: May 26, 2017

(30) Foreign Application Priority Data
Dec. 12, 2014 (JP) 2014-251723
Dec. 12, 2014 (JP) 2014-251724

(51) Int. Cl. A61B 5/024; A61B 5/00

(52) U.S. Cl. CPC A61B 5/02444; A61B 5/721; A61B 5/74; A61B 5/0082; A61B 5/681; A61B 2562/0219

(57) ABSTRACT

A sensor unit, a biological information detection device, an electronic apparatus, a biological information detection method, and the like, capable of acquiring highly accurate pulse wave information on the basis of a plurality of signals having different characteristics. The sensor unit includes a first light emitting portion that emits light toward a subject, a second light emitting portion that emits light toward the subject, and a light receiving portion that receives light from the subject, in which, in a case where a height of a contact position or a contact region with the subject in a position or a region corresponding to the first light emitting portion is indicated by H1, and a height of a contact position or a contact region with the subject in a position or a region corresponding to the second light emitting portion is indicated by H2, a relationship of H1>H2 is satisfied.

### TECHNICAL FIELD

The present invention relates to a sensor unit, a biological information detection device, an electronic apparatus, a biological information detection method, and the like.

### BACKGROUND ART

Since a pulse wave indicates a change in a blood vessel volume, a photoelectric pulse wave sensor can measure a pulse wave by recognizing a change in a blood volume of a measurement target part. However, a blood volume of a measured part changes not only due to beating (that is, a pulse wave) of the heart but also due to movement of a human body (hereinafter, also referred to as body movement). Thus, when a pulse wave is measured by using a photoelectric pulse wave sensor, there is a case where noise caused by body movement is included in a wave motion in the process of propagation from the heat to a measured part. In other words, since blood is a fluid, and a blood vessel is elastic, there is a case where a flow of blood caused by body movement causes a change in a blood volume, and thus a false pulsation is measured.

A pulse wave measurement device which performs such a calculation process for removing a noise component caused by body movement has been developed. For example, PTL 1 discloses a technique in which light beams with different wavelengths are applied, reflected light beams thereof are simultaneously measured, and a pulsation component is extracted from measured values. This technique uses the fact that there are different light absorption characteristics between dominant oxygenated hemoglobin in arterial blood and dominant reduced hemoglobin in venous blood.

### CITATION LIST

### Patent Literature

PTL 1: JP-A-55-120858

### SUMMARY OF INVENTION

### Technical Problem

However, irradiation light beams having different wavelengths used for a sensor which detects reflected light so as to measure a pulse wave also have different depths of permeation into a living body. Thus, in the technique disclosed in PTL 1, a difference in absorbance occurring between a plurality of sensors also includes the influence of a difference in a permeation depth between light beams with different wavelengths, and thus it is hard to reduce noise caused by body movement.

According to some aspects of the invention, it is possible to provide a sensor unit, a biological information detection device, an electronic apparatus, a biological information detection method, and the like, capable of detecting a plurality of signals which have correlation to some extent and have different characteristics.

According to some aspects of the invention, it is possible to provide a sensor unit, a biological information detection device, an electronic apparatus, a biological information detection method, and the like, capable of acquiring highly accurate pulse wave information on the basis of signals having different characteristics, acquired by using light beams from a plurality of light emitting portions.

### Solution to Problem

An aspect of the invention relates to a sensor unit including a first light emitting portion that emits light toward a subject; a second light emitting portion that emits light toward the subject; and a light receiving portion that receives light from the subject, in which, in a case where a height of a contact position or a contact region with the subject in a position or a region corresponding to the first light emitting portion is indicated by H1, and a height of a contact position or a contact region with the subject in a position or a region corresponding to the second light emitting portion is indicated by H2, a relationship of H1>H2 is satisfied.

In the aspect of the invention, in the sensor unit including a plurality of light emitting portions, a difference is set in a height (specifically, a height of a portion which comes into contact with a subject) in a position or a region corresponding to each light emitting portion. Thus, since a difference occurs in pressure applied to a subject in a contact position or a contact region, it is possible to set a difference between signal characteristics on the basis of a light reception result based on light from the first light emitting portion and a light reception result based on light from the second light emitting portion.

In the aspect of the invention, H1 may indicate a height of a contact position or a contact region with the subject in an arrangement region of the first light emitting portion, and H2 may indicate a height of a contact position or a contact region with the subject in an arrangement region of the second light emitting portion.

With this configuration, it is possible to dispose the light emitting portion with a position or a region corresponding to the light emitting portion as a reference.

In the aspect of the invention, in a case where a height to a surface of the first light emitting portion on the subject side is indicated by HA1, and a height to a surface of the second light emitting portion on the subject side is indicated by HA2, a relationship of HA1>HA2 may be satisfied so that the relationship of H1>H2 is satisfied.

With this configuration, a difference is set in a height to a surface of the light emitting portion on the subject side, and thus it is possible to set a difference in a height to a contact position or a contact region.

In the aspect of the invention, the second light emitting portion and the light receiving portion may be provided on a board, and a height adjustment member may be provided between the first light emitting portion and the board.

With this configuration, it is possible to set a height difference by using the height adjustment member.

In the aspect of the invention, the height adjustment member may be a second board, and an external connection terminal of the first light emitting portion may be connected to a connection terminal provided on the board via a through hole of the second board.

With this configuration, it is possible to use the second board as the height adjustment member.

In the aspect of the invention, an external connection terminal of the first light emitting portion may be connected to a connection terminal provided on the board via a wire.

With this configuration, the first light emitting portion can be connected to the board (main board) by using the wire, and thus it is possible to use a member (for example, an insulator) which cannot perform electrical connection by itself as the height adjustment member.

In the aspect of the invention, in a case where a direction directed toward the subject from the sensor unit is set to a first direction when biological information is detected, a length of the first light emitting portion in the first direction is indicated by LH1, and a length of the second light emitting portion in the first direction is indicated by LH2, a relationship of LH1>LH2 may be satisfied so that the relationship of H1>H2 is satisfied.

With this configuration, a length (a height or a thickness) of the light emitting portion is set to differ, and thus it is possible to set a difference in a height to a contact position or a contact region.

In the aspect of the invention, the sensor unit may further include a first member provided between at least the first light emitting portion and the light receiving portion; and a second member provided between at least the second light emitting portion and the light receiving portion, and, in a case where a height of the first member is indicated by HC1, and a height of the second member is indicated by HC2, a relationship of HC1>HC2 may be satisfied so that the relationship of H1>H2 is satisfied.

With this configuration, a predetermined member corresponding to the first light emitting portion and another member corresponding to the second light emitting portion are provided, a difference is set between heights of the members, and thus it is possible to set a difference in a height to a contact position or a contact region.

In the aspect of the invention, the sensor unit may further include a light transmissive member that is provided at a position located further toward the subject side than the first light emitting portion, transmits light from the subject therethrough, and comes into contact with the subject so as to apply pressure thereto when biological information of the subject is measured, and, in a case where a height of the light transmissive member in a position or a region corresponding to the first light emitting portion is indicated by HD1, a relationship of HD1>H2 may be satisfied so that the relationship of H1>H2 is satisfied.

With this configuration, the light transmissive member is provided on the subject side of at least the first light emitting portion, a height of the light transmissive member is increased, and thus it is possible to set a difference in a height to a contact position or a contact region.

In the aspect of the invention, the light transmissive member may be provided at a position located further toward the subject side than the second light emitting portion, transmit light from the subject therethrough, and come into contact with the subject so as to apply pressure thereto when biological information of the subject is measured, and, in a case where a height of the light transmissive member in a position or a region corresponding to the second light emitting portion is indicated by HD2, a relationship of HD1>HD2 may be satisfied so that the relationship of H1>H2 is satisfied.

With this configuration, the light transmissive member can also be provided on the subject side of the second light emitting portion, a height difference is set between a height of the light transmissive member in the first light emitting portion and a height of the light transmissive member in the second light emitting portion, and thus it is possible to set a difference in a height to a contact position or a contact region.

In the aspect of the invention, in a case where a height of a contact position or a contact region with the subject in a position or a region corresponding to the light receiving portion is indicated by H3, a relationship of H1≧H3≧H2 (here, a case of H1=H3=H2 is excluded) may be satisfied.

With this configuration, it is possible to use a structure in which not only heights corresponding to the first light emitting portion and the second light emitting portion but also a height corresponding to the light receiving portion is taken into consideration.

In the aspect of the invention, the light receiving portion may receive light from the subject in a case where pressure applied to a measurement part of the subject is first pressure, and light from the subject in a case where the pressure applied to the measurement part of the subject is second pressure lower than the first pressure.

With this configuration, it is possible to receive a plurality of light beams having different pressure states in the light receiving portion.

In the aspect of the invention, a height of the contact region with the subject may be an average height of heights at respective points included in the contact region.

With this configuration, it is possible to use, specifically, an average height in the region as a “height of the region”.

Another aspect of the invention relates to a biological information detection device including the sensor unit.

In the aspect of the invention, the biological information detection device may further include a processing unit that performs a biological information detection process on the basis of a first light reception result which is a light reception result of light from the subject, corresponding to light from the first light emitting portion, and a second light reception result which is a light reception result of light from the subject, corresponding to light from the second light emitting portion.

With this configuration, it is possible to detect biological information by using a light reception result based on light from each light emitting portion.

Another aspect of the invention relates to a biological information detection device including at least a light emitting portion that emits light toward a subject; at least a light receiving portion that receives light from the subject; and a processing unit that performs a biological information detection process on the basis of a detection signal output from the light receiving portion, in which the processing unit performs the biological information detection process on the basis of a first detection signal which is the detection signal in a case where pressure applied to a measurement part of the subject is first pressure, and a second detection signal which is the detection signal in a case where the pressure applied to the measurement part of the subject is second pressure lower than the first pressure.

In the aspect of the invention, biological information is detected on the basis of the first detection signal acquired in a state in which the first pressure is applied, and the second detection signal acquired in a state in which the second pressure is applied. Since characteristics of light reception results (detection signals) are different from each other if pressure states are different from each other, it is possible to detect biological information with high accuracy by using a plurality of signals having different characteristics.

In the aspect of the invention, the processing unit may perform the biological information detection process on the basis of the first detection signal in the light receiving portion at a first timing, and the second detection signal in the light receiving portion at a second timing which is different from the first timing.

With this configuration, it is possible to set light reception timings in the light receiving portion to be different from each other in relation to the first detection signal and the second detection signal.

In the aspect of the invention, the biological information detection device may further include a first light emitting portion and a second light emitting portion as the light emitting portion, and the processing unit may perform the biological information detection process on the basis of the first detection signal in the light receiving portion at the first timing, based on light emission of the first light emitting portion, and the second detection signal in the light receiving portion at the second timing, based on light emission of the second light emitting portion.

Consequently, it is possible to acquire a detection signal at an appropriate timing by controlling a light reception timing in the light receiving portion, corresponding to light from each light emitting portion by using two light emitting portions.

In the aspect of the invention, there may be a configuration in which, in a case where the second timing is a timing subsequent to the first timing, a third timing is a timing subsequent to the second timing, and a fourth timing is a timing subsequent to the third timing, the processing unit acquires the first detection signal in the light receiving portion at the first timing and the third timing, and acquires the second detection signal in the light receiving portion at the second timing and the fourth timing.

With this configuration, the first detection signal and the second detection signal can be alternately acquired in the light receiving portion at at least four timings, and thus the two detection signal having different characteristics can be acquired at timings temporally close to each other.

In the aspect of the invention, the processing unit may perform the biological information detection process on the basis of the first detection signal in the light receiving portion at the first timing, and the second detection signal in the light receiving portion at the first timing.

With this configuration, a biological information detection process can be performed on the basis of detection signals in which measurement conditions (pressure) are different from each other, and thus it is possible to detect biological information with high accuracy.

In the aspect of the invention, the processing unit may perform a correction process on the first detection signal on the basis of the second detection signal, and may perform the biological information detection process on the basis of the corrected first detection signal.

With this configuration, it is possible to use the second detection signal for a correction process on the first detection signal.

In the aspect of the invention, the processing unit may perform a correction process on the second detection signal on the basis of the first detection signal, and may perform the biological information detection process on the basis of the corrected second detection signal.

With this configuration, it is possible to use the first detection signal for a correction process on the second detection signal.

In the aspect of the invention, the processing unit may perform a body movement noise reduction process of reducing body movement noise included in the detection signal as the correction process.

With this configuration, the body movement noise reduction process can be performed as the correction process so that the influence of body movement noise is reduced, and thus it is possible to detect biological information with high accuracy.

Another aspect of the invention relates to an electronic apparatus including the sensor unit.

Another aspect of the invention relates to an electronic apparatus including the biological information detection device.

Another aspect of the invention relates to a biological information detection method for a biological information detection device including at least one light emitting portion that emits light toward a subject, and at least one light receiving portion that receives light from the subject, the method including performing the biological information detection process on the basis of a first detection signal which is the detection signal in a case where pressure applied to a measurement part of the subject is first pressure, and a second detection signal which is the detection signal in a case where the pressure applied to the measurement part of the subject is second pressure lower than the first pressure.

### BRIEF DESCRIPTION OF DRAWINGS

FIGS. 1(A) to 1(C) illustrate configuration examples of a sensor unit according to the present embodiment.

FIGS. 2(A) and 2(B) are exterior views of a biological information detection device according to the present embodiment.

FIG. 3 is an exterior view of the biological information detection device according to the present embodiment.

FIG. 4 is a diagram for explaining mounting of the biological information detection device and communication with a terminal apparatus.

FIG. 5 is a functional block diagram of the biological information detection device.

FIG. 6 is a perspective view illustrating an exterior of the sensor unit.

FIG. 7 is a plan view illustrating an example in which a light receiving portion and first and second light emitting portions are disposed.

FIG. 8 is a diagram exemplifying a change in absorbance with respect to pressure.

FIG. 9 is a diagram exemplifying a change in body movement noise sensitivity with respect to pressure.

FIGS. 10(A) and 10(B) are diagrams for explaining the extent to which an MN ratio (SN ratio) is improved through a noise reduction process in cases where a pressure difference is set and is not set.

FIG. 11 is a diagram illustrating a relationship between cuff pressure, and DC components and AC components detected in the light receiving portion.

FIGS. 12(A) to 12(C) illustrate configuration examples of the sensor unit according to the present embodiment.

FIGS. 13(A) and 13(B) are diagrams for explaining positions or regions corresponding to the first and second light emitting portions.

FIGS. 14(A) and 14(B) are respectively a plan view and a sectional view illustrating an example in which a frame portion is disposed.

FIGS. 15(A) and 15(B) are plan views illustrating another example in which the frame portion is disposed.

FIG. 16 is a diagram for explaining the influence which a distance between the light emitting portion and the light receiving portion exerts on a light permeation depth.

FIG. 17 is a diagram illustrating a relationship between a distance between the light emitting portion and the light receiving portion, and the intensity of a detection signal.

FIG. 18 is a diagram illustrating a relationship between a distance between the light emitting portion and the light receiving portion, and a measurement distance in a depth direction.

FIGS. 19(A) and 19(B) are diagrams for explaining a case where a difference is set in a distance between the light emitting portion and the light receiving portion.

FIG. 20 illustrates examples of a light emission timing in each light emitting portion and a light reception timing in the light receiving portion.

FIGS. 21(A) and 21(B) are diagrams for explaining a body movement noise reduction process using a second detection signal.

FIG. 22 is a diagram for explaining adaptive filter processing.

FIG. 23 is a diagram for explaining a flow of signal processing.

### DESCRIPTION OF EMBODIMENTS

Hereinafter, the present embodiment will be described. The present embodiment described below is not intended to improperly limit the content of the invention disclosed in the claims. It cannot be said that all constituent elements described in the present embodiment are essential constituent elements of the invention.

### 1. Method of Present Embodiment

First, a method of the present embodiment will be described. As described above, in a case where biological information such as pulse wave information is detected by using a photoelectric sensor, noise due to body movement is problematic. Thus, in order to detect biological information with high accuracy, it is necessary to reduce body movement noise according to a predetermined method. Here, the body movement noise, which is noise caused by body movement of a subject, includes physical noise caused by a change in velocity, acceleration, an action, an attitude, and the like due to an action of a subject, and optical noise caused by a change in a measurement environment of the photoelectric sensor, such as incidence of external light or deviation in a measurement position, due to a change in a mounting state resulting from an action of the subject, and indicates at least one thereof.

In order to reduce the body movement noise, a component corresponding to a pulse signal is maintained if at all possible in a detection signal from the photoelectric sensor, and a component corresponding to the body movement noise is reduced (removed in a narrow sense). In other words, in a process of reducing the body movement noise, it is necessary to identify a signal component corresponding to the body movement noise.

In contrast, there is a method of reducing body movement noise by using a motion sensor. Since the motion sensor is a sensor detecting a motion of a user (a person wearing the biological information detection device), a signal corresponding to body movement, that is, a signal corresponding to body movement noise can be acquired by using the motion sensor. The motion sensor here may be, for example, an acceleration sensor, a gyro sensor, or an atmospheric pressure sensor.

Also in the present embodiment, a method of reducing body movement noise by using the motion sensor may also be used, but the present applicant proposes another body movement noise reducing method. Specifically, a signal including a lot of body movement noise is acquired by using a second light emitting portion which is different from a first light emitting portion emitting light for detecting a pulse signal.

As described above, a detection signal in the photoelectric sensor includes body movement noise. By using this fact, a signal corresponding to the second light emitting portion is set so that the sensitivity for a pulse signal is low, and the sensitivity of body movement noise is high, and thus a detection signal mainly including body movement noise can be acquired.

If a signal corresponding to body movement noise can be detected on the basis of light from the second light emitting portion, the body movement noise can be reduced by removing (reducing) a component corresponding to a detection signal (second detection signal) based on light from the second light emitting portion, from a detection signal (first detection signal) based on light from the first light emitting portion. In this case, since the sensitivity of a pulse signal is low in the second detection signal, a pulse component included in the first detection signal is not excessively reduced.

However, in order to perform this process, it is necessary to make characteristics (for example, frequency characteristics) of body movement noise included in the first detection signal and the second detection signal match each other, or sufficiently similar to each other. In other words, it is necessary to set a difference in signal characteristics and also to cause a high correlation between the two detection signals to be maintained so that the first detection signal mainly includes a pulse signal, and the second detection signal mainly includes body movement noise.

In the technique disclosed in PTL 1, a plurality of light receiving portions are provided, and signals having different characteristics are acquired, but, frequency bands of light beams detected by the respective light receiving portions are greatly different from each other. Thus, characteristics of detection signals at the respective light receiving portions can be made different from each other, but a correlation therebetween to some extent is hardly given. This is because, if wavelengths of light are different from each other, permeation depths into a living body are also different from each other, and thus a structure of a blood vessel, a bone, or the like as a detection target differs in the first place.

Thus, in the present embodiment, wavelength bands of light beams to be used are assumed to be the same as each other in a plurality of detection signals. The light beams with the same wavelength band do not indicate wavelengths at which the intensity is the maximum are exactly the same as each other, but indicate that wavelengths at which the intensity is the maximum are included in a predetermined range (for example, a range of the same color). Light beams output from the first light emitting portion and the second light emitting portion are light beams with wavelength bands which are all included in, for example, a range of 470 nm to 610 nm. More specifically, light beams output from the first light emitting portion and the second light emitting portion are light beams with wavelength bands included in a range of 520 nm to 570 nm. The light beams with the wavelength bands are more easily reflected at hemoglobin in blood than light beams with other wavelength bands. Specifically, the light beams are, for example, green light (500 nm to 570 nm).

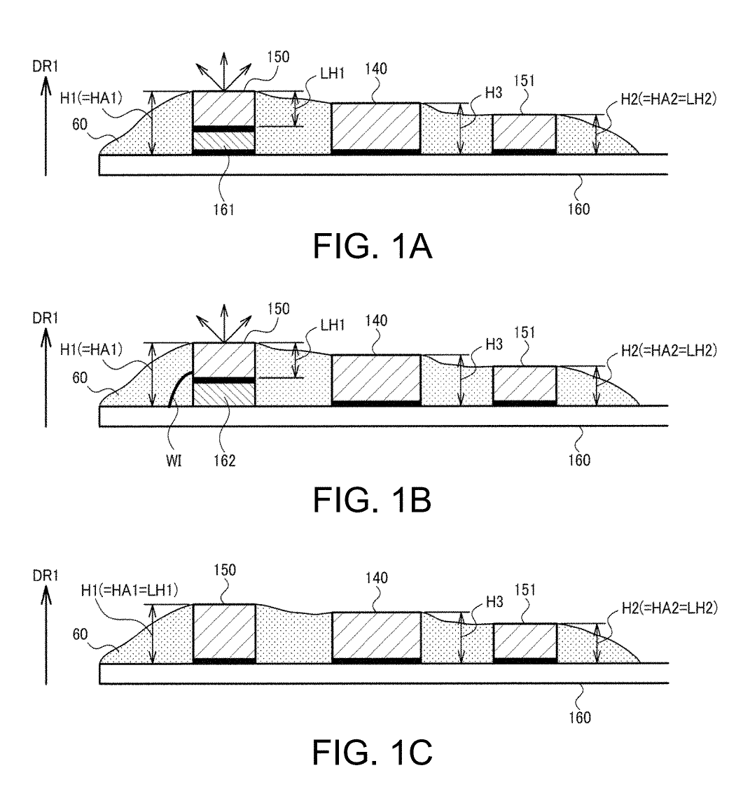

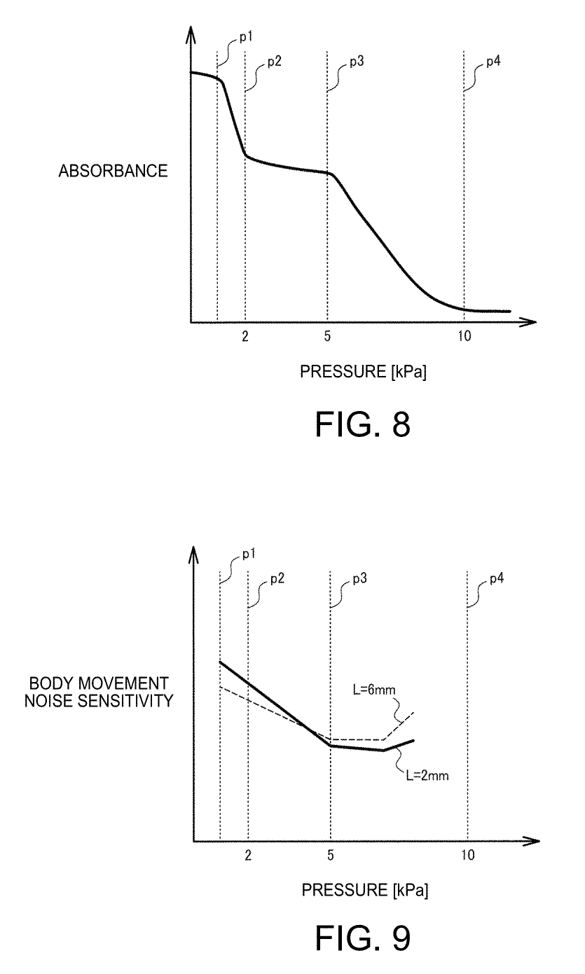

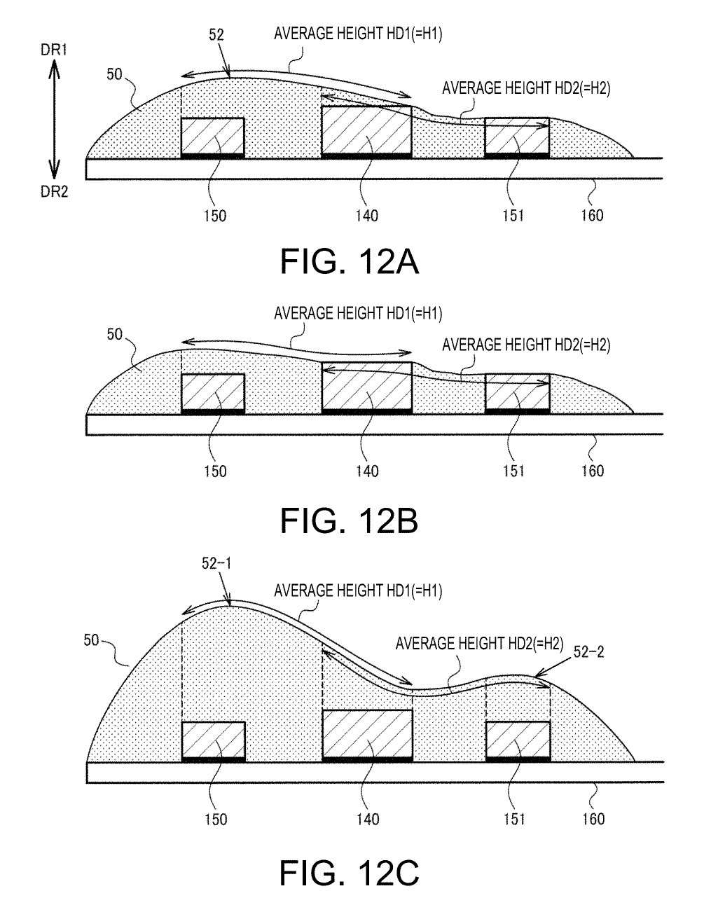

Hereinafter, in the present specification, as described above, a description will be made of specific configurations of a sensor unit and a biological information detection device allowing the requirement that detection signals have a correlation to some extent and have different characteristics, to be satisfied. As will be described later with reference to FIGS. 8, 9 and 17, and the like, it can be seen that the sensitivity for a pulse signal or the sensitivity for body movement noise changes according to pressure applied to a subject, or a distance between a light emitting portion and a light receiving portion. The pressure applied to the subject can be adjusted by using a height of a member which comes into contact with the subject in a position or a region corresponding to the light emitting portion. The member which comes into contact with the subject may be a surface of a light emitting portion as will be described later with reference to FIGS. 1(A) to 1(C), and may be a light transmissive member 50 which is provided further toward the subject side than a light emitting portion as will be described later with reference to FIGS. 12(A) to 12(C).

Hereinafter, a description will be made of a case where body movement noise reduction process can be realized by providing a pressure difference, and a description will be made of a detailed structure for providing the pressure difference. Specifically, a description will be made of a method of setting a height of a contact position (region) with a subject at a position (region) corresponding to a light emitting portion, and a distance between a light emitting portion and a light receiving portion, and specific configurations of a sensor unit and a biological information detection device having a height and a distance which are appropriately set according to the method.

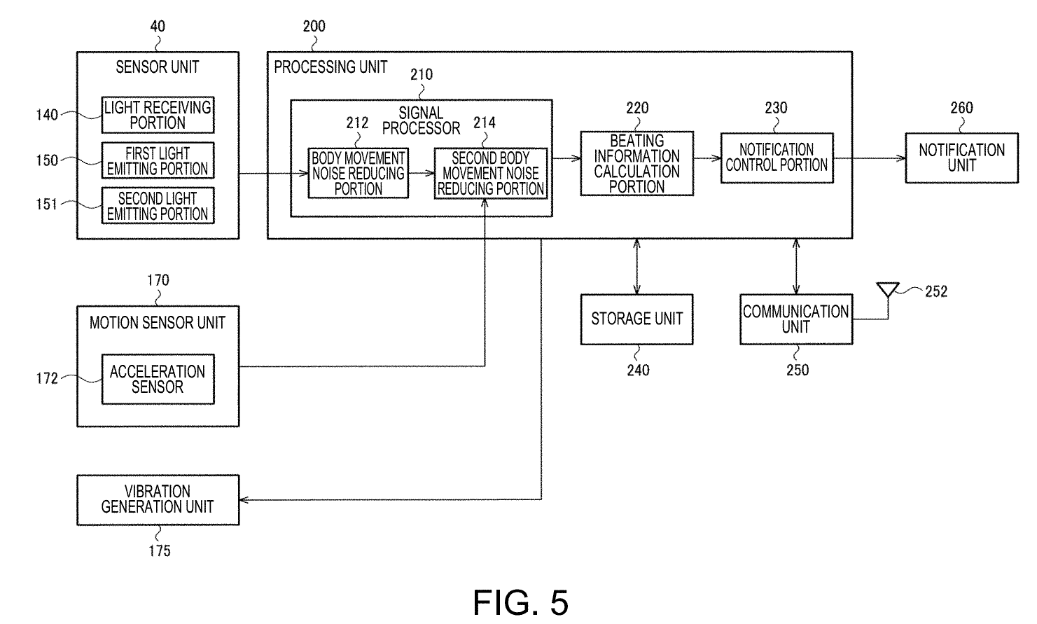

As mentioned above, the biological information detection device according to the present embodiment includes at least one light emitting portion which emits light toward a subject, at least one light receiving portion receiving light from the subject, and a processing unit 200 which performs a biological information detection process on the basis of a detection signal (a light reception result from the light receiving portion) output from the light receiving portion. A biological information detection device 400 may have, for example, a configuration which will be described later with reference to FIG. 5. The processing unit 200 performs a biological information detection process on the basis of a first detection signal which is a detection signal in a case where pressure applied to a measurement part of a subject is first pressure P1 and a second detection signal which is a detection signal in a case where pressure applied to a measurement part of the subject is second pressure P2 lower than the first pressure P1.

Regarding a specific configuration of a sensor unit 40 for realizing a pressure difference, structures or the like illustrated in FIGS. 1(A) to 1(C) may be employed. For example, the sensor unit 40 illustrated in FIG. 1(A) includes a first light emitting portion 150 emitting light toward a subject, a second light emitting portion 151 emitting light toward the subject, and a light receiving portion 140 receiving light from the subject. In a case where a height of a contact position or a contact region with the subject in a position or a region corresponding to the first light emitting portion 150 is indicated by H1, and a height of a contact position or a contact region with the subject in a position or a region corresponding to the second light emitting portion 151 is indicated by H2, a relationship of H1>H2 is satisfied.

Here, there may be various methods of defining a height, and, for example, a predetermined reference surface maybe set, and a distance to the reference surface (specifically, a length to a target position in a direction perpendicular to the reference surface with the reference surface as a starting point) may be set as a height. Here, in order to apply considerable pressure to a predetermined portion, the portion is required to protrude further toward a subject side than other portions of the biological information detection device 400 (the sensor unit 40 in a narrow sense). When this matter is taken into consideration, the predetermined reference surface is a surface in which a distance to the subject is uniform when the biological information detection device 400 is mounted, and, if the subject is a flat surface, the reference surface is a flat surface which is parallel to the flat surface. When the biological information detection device 400 is mounted, it is insignificant to mount the device so that the sensor unit 40 is inclined with respect to the subject, and thus it is expected that a surface used as a reference of the sensor unit 40, for example, a board (main board) 160 on which the light emitting portion (LED) or the light receiving portion (PD) is parallel to the subject surface or is substantially parallel thereto. Actually, a subject is not an ideal flat surface, and a state in which the biological information detection device 400 is mounted changes due to a motion state of a user, but there is no big problem in that a board surface on which elements of the sensor unit 40 are mounted is taken into consideration as the “predetermined reference surface”. Therefore, the predetermined reference surface in the present embodiment may be, for example, a surface of the board 160 or a surface parallel thereto. The predetermined reference surface here is not limited to taking into consideration a physical surface on which a certain member is provided, and may be a virtual plane.

The positions or the regions corresponding to the first light emitting portion 150 and the second light emitting portion 151 may be central positions of the respective light emitting portions, may be regions where the respective light emitting portions are disposed, and may be regions including regions where the respective light emitting portions are disposed, and specific examples thereof will be described later.

The contact position or the contact region is a position or a region where the biological information detection device 400 (sensor unit 40) comes into contact with a subject. It is expected that the biological information detection device 400 comes into contact with the subject at a plurality of positions or in a region having some area, but, here, above all, positions or regions corresponding to the first light emitting portion 150 and the second light emitting portion 151 are taken into consideration. In a narrow sense, an intersection between a straight line in a direction perpendicular to a reference surface from positions or regions corresponding to the first light emitting portion 150 and the second light emitting portion 151, and the subject, may be used. Specific examples of contact positions or contact regions at the positions or in the regions corresponding to the first light emitting portion 150 and the second light emitting portion 151, and heights thereof, will be described later. In the following present specification, for simplification, a contact position will be fundamentally described, but, a contact position in the following description may be extended to a contact region.

For simplification, FIGS. 1(A) to 1(C) schematically illustrate a configuration of the biological information detection device according to the present embodiment, and dimensions or scales in the figures are different from actual ones. This is also the same for FIGS. 12(A) to 12(C), and the like.

In the above-described way, pressure states can be made different from each other in a first light reception result (first detection signal) which is a light reception result corresponding to light emitted from the first light emitting portion 150 and a second light reception result (second detection signal) which is a light reception result corresponding to light emitted from the second light emitting portion 151. Specifically, the first detection signal corresponds to the first pressure P1, and the second detection signal corresponds to the second pressure P2 satisfying P2<P1.

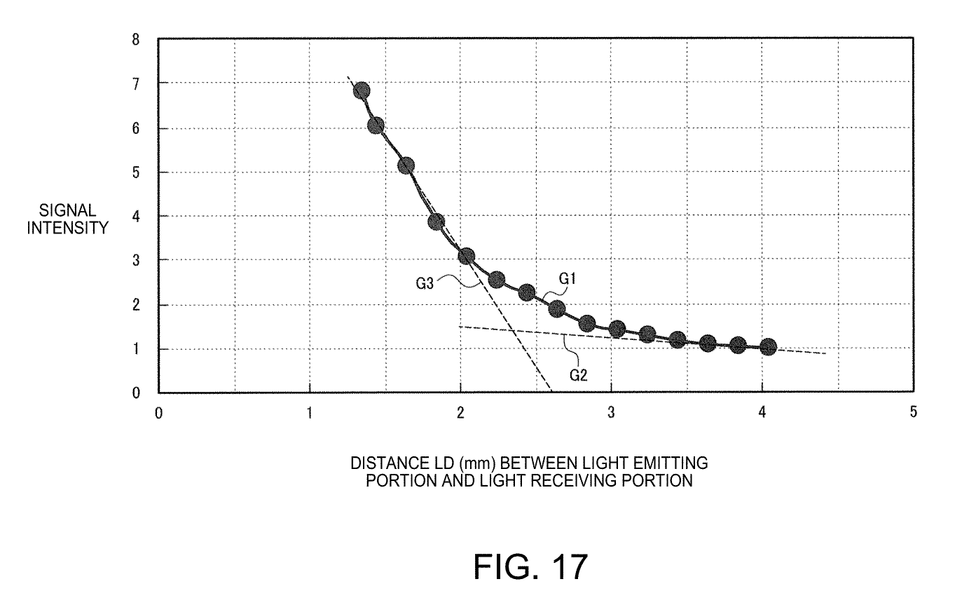

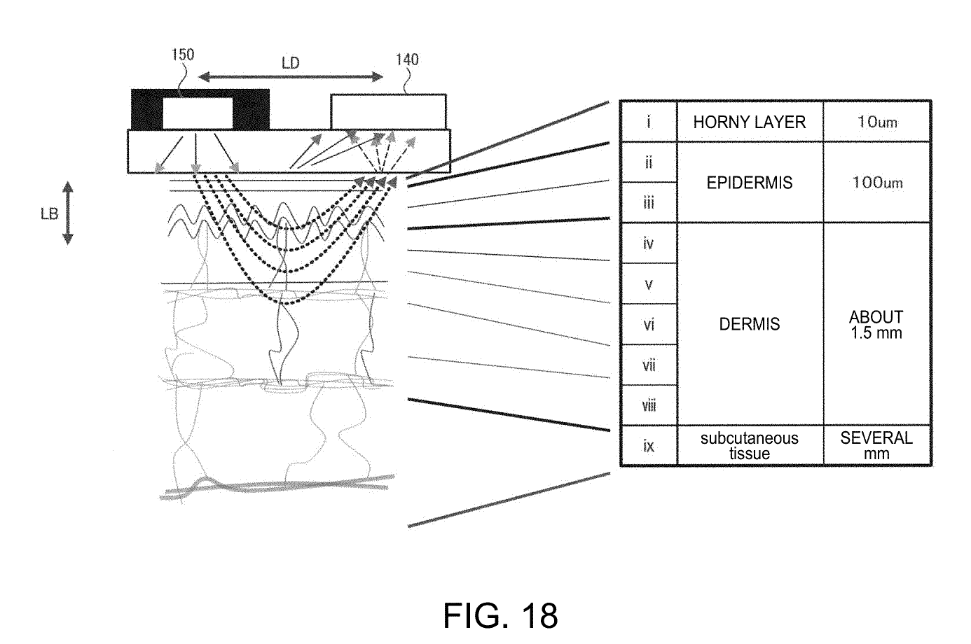

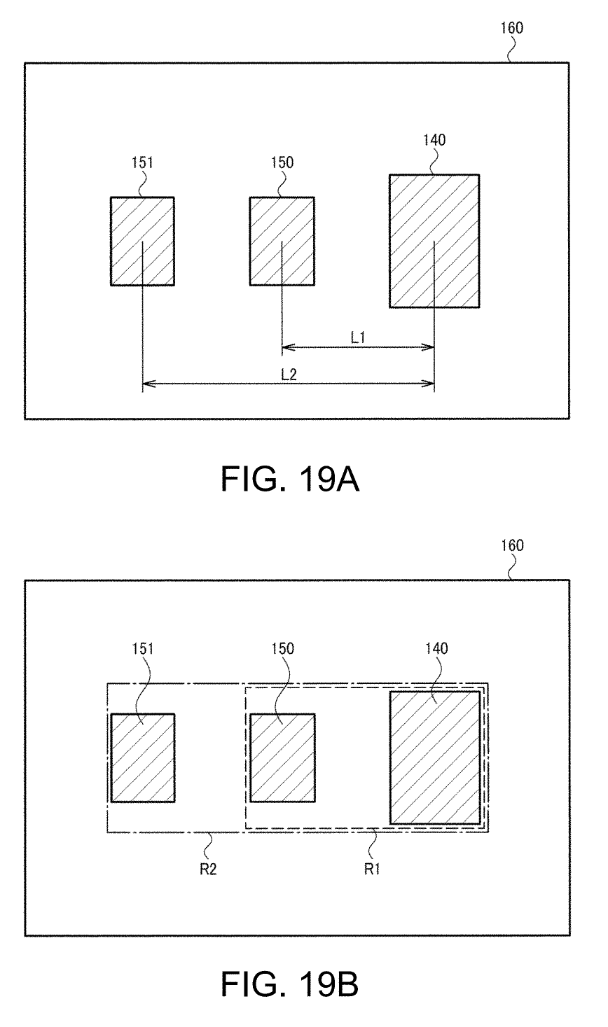

In the sensor unit 40 according to the present embodiment, in a case where a distance between the first light emitting portion 150 and the light receiving portion 140 is indicated by L1, and a distance between the second light emitting portion 151 and the light receiving portion 140 is indicated by L2, a relationship of L1<L2 may be satisfied as illustrated in FIG. 19(A). Details thereof will be described later with reference to FIG. 17 or 18.

In the above-described way, a difference can be provided to at least one of heights of contact positions with a subject in positions or regions corresponding to the respective light emitting portions and distances between the light receiving portion and the respective light emitting portions, and, thus, as described above, it is possible to mainly detect a pulse signal on the basis of light from the first light emitting portion 150, and to mainly detect body movement noise on the basis of light from the second light emitting portion 151. Therefore, it is possible to perform body movement noise reduction process using the second detection signal with respect to the first detection signal, or to obtain highly accurate biological information on the basis of the first detection signal after the body movement noise reduction process is performed.

As illustrated in FIGS. 1(A) to 1(C), and the like, a distance between the light emitting portion and the light receiving portion 140 may be the same in two sets of photoelectric sensors. In other words, a structure in which a difference is set in a distance between the light emitting portion and the light receiving portion 140 is not essential, and may be used as necessary.

2. Configuration Example of Biological Information Detection Device and the Like

2.1 Entire Configuration Example of Biological Information Detection Device

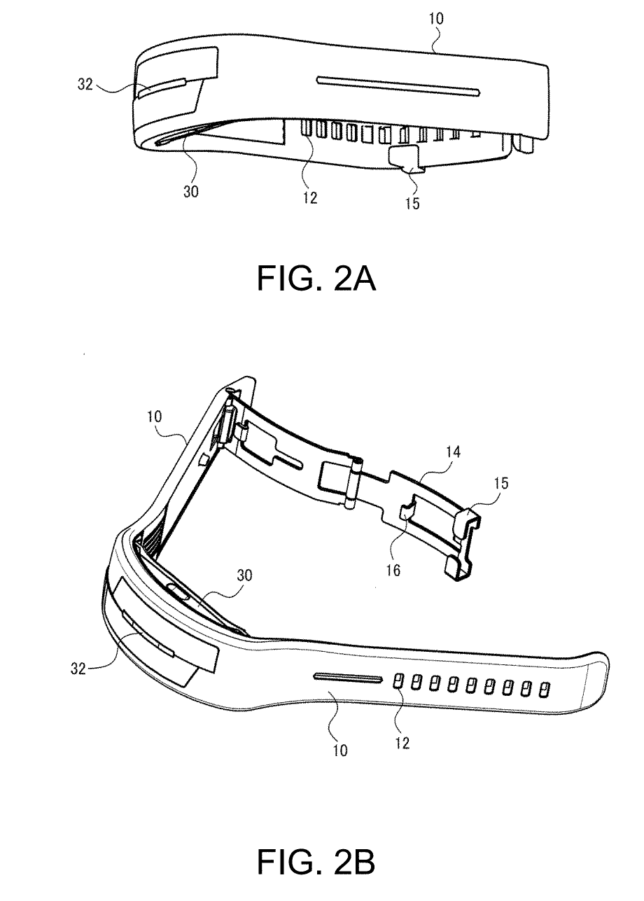

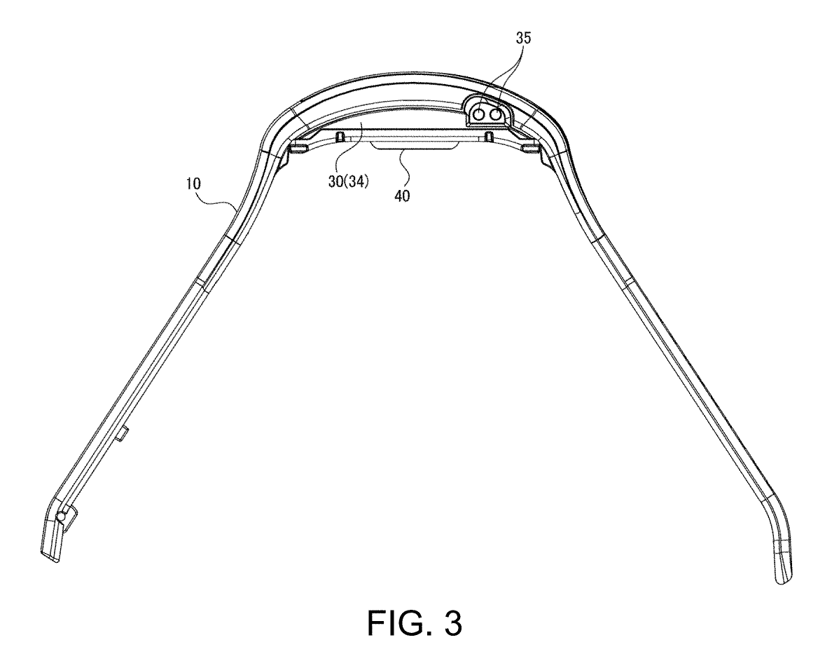

FIGS. 2(A) and 2(B) and FIG. 3 are exterior views of the biological information detection device 400 (biological information measurement device) according to the present embodiment. FIG. 2(A) is a view in which the biological information detection device 400 is viewed from a front direction side, FIG. 2(B) is a view in which the biological information detection device is viewed from an upper direction side, and FIG. 3 is a view in which the biological information detection device is viewed from a lateral direction side.

As illustrated in FIGS. 2A to 3, the biological information detection device 400 of the present embodiment including a band 10, a case 30, and the sensor unit 40. The case 30 is attached to the band 10. The sensor unit 40 is provided in the case 30. The biological information detection device 400 includes the processing unit 200 as illustrated in FIG. 5 which will be described later. The processing unit 200 is provided in the case 30, and detects biological information on the basis of a detection signal from the sensor unit 40. A configuration of the biological information detection device 400 of the present embodiment is not limited to the configuration illustrated in FIGS. 2A to 3, and may be variously modified by omitting some constituent elements, replacing constituent elements with other constituent elements, or adding other constituent elements thereto.

The band 10 is wound on the wrist of a user so as to mount the biological information detection device 400 on the wrist. The band 10 has band holes 12 and a buckle 14. The buckle 14 is provided with a band insertion portion 15 and a protrusion 16. The user inserts one end side of the band 10 into the band insertion portion 15 of the buckle 14, and inserts the protrusion 16 of the buckle 14 into the band hole 12 of the band 10, so as to mount the biological information detection device 400 on the wrist. In this case, the magnitude of the pressure for the sensor unit 40 (pressure against a wrist surface) which will be described later is adjusted depending on the band holes 12 into which the protrusion 16 is inserted. The band 10 may be provided with a clasp instead of the buckle 14.

The case 30 corresponds to a main body of the biological information detection device 400. The case 30 is provided with various constituent elements of the biological information detection device 400, such as the sensor unit 40 and the processing unit 200 therein. In other words, the case 30 is a casing which stores these constituent elements. The case 30 has, for example, a top case 34 and a bottom case 36. The case 30 may not have an aspect of being divided into the top case 34 and the bottom case 36.

The case 30 is provided with a light emission window 32. The light emission window 32 is formed of a light transmissive member. The case 30 is provided with a light emitting portion (which is an LED and is the light emitting portion for notification, different from the light emitting portion 150 of the light detection unit) mounted on a flexible board, and light from the light emitting portion is emitted to the outside of the case 30 via the light emission window 32.

As illustrated in FIG. 3, the case 30 is provided with terminal portions 35. If the biological information detection device 400 is attached to a cradle (not illustrated), terminal portions of the cradle are electrically connected to the terminal portions 35 of the case 30. Consequently, a secondary battery (battery) provided in the case 30 can be charged. A terminal such as a micro-USB maybe provided in the biological information detection device 400, and the battery can be charged by using a micro-USB cable.

The sensor unit 40 detects biological information such as a pulse wave of the subject. For example, as illustrated in FIG. 1(A), the sensor unit 40 includes the light receiving portion 140, the first light emitting portion 150, and the second light emitting portion 151. The sensor unit 40 may include the light transmissive member 50 as will be described later with reference to FIGS. 12(A) to 12(C). The first light emitting portion 150 and the second light emitting portion 151 emit light in a state in which pressure is applied at a contact position with the subject, the light receiving portion 140 receives light reflected by the subject (blood vessel), and light reception results are output to the processing unit 200 as the first detection signal and the second detection signal.

The processing unit 200 performs a noise reduction process on the first detection signal on the basis of the second detection signal from the sensor unit 40, and detects biological information such as a pulse wave on the basis of the first detection signal having undergone the noise reduction process. Biological information as a detection target of the biological information detection device 400 of the present embodiment is not limited to a pulse wave (pulse rate), and the biological information detection device 400 maybe a device which detects biological information (for example, oxygen saturation in blood, a body temperature, and a heartbeat) other than a pulse wave.

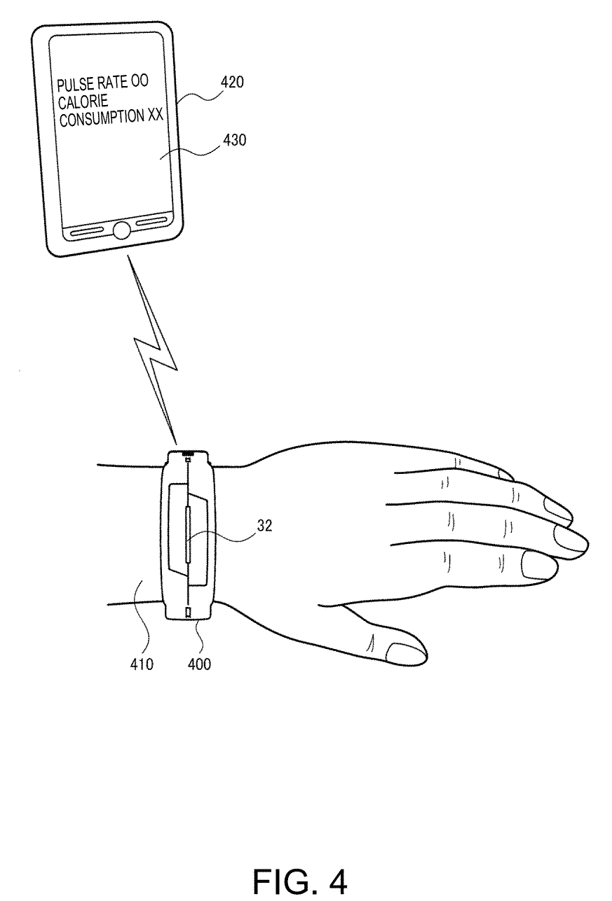

FIG. 4 is a diagram for explaining mounting of the biological information detection device 400 and communication with a terminal apparatus 420. As illustrated in FIG. 4, the user as a subject mounts the biological information detection device 400 on the wrist 410 like a wristwatch. As illustrated in FIG. 3, the sensor unit 40 is provided on a surface of the case 30 on the subject side. Therefore, if the biological information detection device 400 is mounted, the sensor unit 40 comes into contact with the skin surface of the wrist 410 so as to apply pressure thereto, the first light emitting portion 150 and the second light emitting portion 151 of the sensor unit 40 emit light in this state, the light receiving portion 140 receives reflected light, and thus biological information such as a pulse wave is detected. Amounting part may be an ankle, a finger, an upper arm, or the like.

The biological information detection device 400 and the terminal apparatus 420 are communicably connected to each other, and can perform transmission and reception of data. The terminal apparatus 420 is a portable communication terminal such as a smart phone, a mobile phone, or a feature phone. Alternatively, the terminal apparatus 420 may be an information processing terminal such as a tablet computer. For example, near field communication (NFC) such as Bluetooth (registered trademark) maybe employed in communication connection between the biological information detection device 400 and the terminal apparatus 420. As mentioned above, since the biological information detection device 400 and the terminal apparatus 420 are communicably connected to each other, various pieces of information such as a pulse rate or calorie consumption can be displayed on a display unit 430 (an LCD or the like) of the terminal apparatus 420. In other words, it is possible to display various pieces of information obtained on the basis of detection signals from the sensor unit 40. A calculation process on information such as a pulse rate or calorie consumption may be performed by the biological information detection device 400, and at least a part thereof may be performed by the terminal apparatus 420.

The biological information detection device 400 is provided with the light emission window 32, and thus notifies the user of various pieces of information through light emission of the light emitting portion for notification (lighting or blinking). For example, in a case where a pulse rate enters a fat combustion zone or leaves the fat combustion zone, the user is notified thereof through light emission of the light emitting portion via the light emission window 32. If a mail or the like is received in the terminal apparatus 420, the terminal apparatus 420 notifies biological information detection device 400 thereof. If the light emitting portion of the biological information detection device 400 emits light, the user is notified of the reception of the mail or the like.

In the examples illustrated in FIGS. 2A to 4, the biological information detection device 400 is not provided with a display unit such as an LCD, and information which is required to be sent with text or numbers is displayed on the display unit 430 of the terminal apparatus 420. As mentioned above, in FIG. 4, the user is notified of required minimum information through light emission of the light emitting portion without providing a display unit such as an LED, and thus miniaturization of the biological information detection device 400 is realized. Since a display unit is provided in the biological information detection device 400, it is possible to improve the appearance of the biological information detection device 400. However, there may be the occurrence of a modification in which the biological information detection device 400 is provided with a display unit such as an LCD or an organic EL display.

The method of the present embodiment is not limited to being applied to the biological information detection device 400, and may be applied to the sensor unit 40. A detailed configuration of the sensor unit 40 will be described later. The method of the present embodiment may be applied to an electronic apparatus including the sensor unit 40, or may be applied to an electronic apparatus including the biological information detection device 400.

Another aspect of the invention may be applied to a biological information detection method for a biological information detection device including at least one light emitting portion emitting light toward a subject and at least one light receiving portion receiving light from the subject, the biological information detection method (a method of operating the biological information detection device) including performing a biological information detection process on the basis of a first detection signal which is a detection signal in a case where pressure applied to a measurement part of the subject is first pressure, and a second detection signal which is the detection signal in a case where the pressure applied to the measurement part of the subject is second pressure lower than the first pressure.

2.2 Functional Block Diagram

FIG. 5 is a functional block diagram of the biological information detection device 400 according to the present embodiment. In FIG. 5, the biological information detection device 400 includes the sensor unit 40, a motion sensor unit 170, a vibration generation unit 175, the processing unit 200, a storage unit 240, a communication unit 250, an antenna 252, and a notification unit 260. A configuration of the biological information detection device 400 of the present embodiment is not limited to the configuration illustrated in FIG. 5, and maybe variously modified by omitting some constituent elements, replacing constituent elements with other constituent elements, or adding other constituent elements thereto.

The sensor unit 40 detects biological information such as a pulse wave, and includes the light receiving portion 140, the first light emitting portion 150, and the second light emitting portion 151. However, the sensor unit 40 may include three or more light emitting portions. Here, as illustrated in FIGS. 1(A) to 1(C) and the like, a description has been made of an example in which the light receiving portion 140 is used to be shared by a plurality of light emitting portions, but the number of light receiving portions is not limited to one, and two or more light receiving portions may be provided.

A pulse wave sensor (photoelectric sensor) is implemented by the light receiving portion 140, the first light emitting portion 150, the second light emitting portion 151, and the like. In a case of FIG. 5, a first pulse wave sensor is implemented by the light receiving portion 140 and the first light emitting portion 150, and a second pulse wave sensor is implemented by the light receiving portion 140 and the second light emitting portion 151. The sensor unit 40 outputs signals detected by a plurality of pulse wave sensors as detection signals (pulse wave detection signals).

The motion sensor unit 170 outputs a body movement detection signal as a signal which changes depending on body movement on the basis of sensor information from various motion sensors. The motion sensor unit 170 includes, for example, an acceleration sensor 172 as an example of the motion sensor. The motion sensor unit 170 may include a pressure sensor or a gyro sensor as a motion sensor, a position sensor such as a GPS receiver, and the like.

The processing unit 200 performs, for example, various signal processes or control processes with the storage unit 240 as a work area, and may be implemented by, for example, a processor such as a CPU or a logic circuit such as an ASIC. The processing unit 200 includes a signal processor 210, a beating information calculation portion 220, and a notification control portion 230.

The signal processor 210 performs various signal processes (a filter process and the like), and performs signal processes on, for example, a pulse wave detection signal from the sensor unit 40 or body movement detection signal from the motion sensor unit 170.

For example, the signal processor 210 includes a body movement noise reducing portion 212 and a second body movement noise reducing portion 214. The body movement noise reducing portion 212 performs a body movement noise reduction process of reducing (removing) body movement noise which is noise caused by body movement from the first detection signal of pulse wave detection signals on the basis of the second detection signal. The second body movement noise reducing portion 214 performs a second body movement noise reduction process of reducing body movement noise from the first detection signal on the basis of a body movement detection signal from the motion sensor unit 170. Specifically, the body movement noise reduction process in the body movement noise reducing portion 212 may use a spectrum subtraction method, and the second body movement noise reduction process in the second body movement noise reducing portion 214 may use an adaptive filter or the like. Details of the processes in the body movement noise reducing portion 212 and the second body movement noise reducing portion 214 will be described later. FIG. 5 illustrates a configuration in which the body movement noise reduction process is performed in the body movement noise reducing portion 212, and then the second body movement noise reduction process is performed in the second body movement noise reducing portion 214, but various modifications such as reversing of process order and omission of the process may occur.

The beating information calculation portion 220 performs a beating information calculation process on the basis of a signal or the like from the signal processor 210. The beating information is, for example, information regarding a pulse rate. Specifically, the beating information calculation portion 220 performs a frequency analysis process such as FFT on a pulse wave detection signal having undergone a noise reduction process in the body movement noise reducing portion 212 and the second body movement noise reducing portion 214 so as to obtain a spectrum, and performs a process of setting a representative frequency in the obtained spectrum as a heartbeat frequency. A value obtained by multiplying the obtained frequency by 60 is a pulse rate (heart rate) which is generally used. The beating information is not limited to a pulse rate itself, and may be various pieces of information (for example, a frequency or a cycle of heartbeats, their changes, and the like) indicating the pulse rate. The beating information may be information indicating a beating state, and may be a value indicating a blood volume itself.

The notification control portion 230 controls the notification unit 260. The notification unit 260 (notification device) notifies the user of various pieces of information under the control of the notification control portion 230. For example, the light emitting portion for notification may be used as the notification unit 260. In this case, the notification control portion 230 controls a current flowing through an LED so as to control lighting or blinking of the light emitting portion. The notification unit 260 may a display unit such as an LCD, a buzzer, or the like.

The notification control portion 230 controls the vibration generation unit 175. The vibration generation unit 175 notifies the user of various pieces of information through vibration. The vibration generation unit 175 may be realized by, for example, a vibration motor (vibrator). The vibration motor generates vibration by rotating, for example, an eccentric weight. Specifically, eccentric weights are attached to both ends of a drive shaft (rotor shaft), and thus the motor vibrates. The vibration of the vibration generation unit 175 is controlled by the notification control portion 230. The vibration generation unit 175 is not limited to such a vibration motor, and may be variously modified. For example, the vibration generation unit 175 may be implemented by a piezoelectric element.

Through vibration generated by the vibration generation unit 175, it is possible to perform, for example, a notification of startup when the supply of power is started, a notification of success of initial pulse wave detection, a warning when a state in which a pulse wave cannot be detected continuously occurs for a predetermined time, a notification during movement to a fat combustion zone, a warning when a battery voltage is reduced, a notification of wake-up alarm, or a notification of a mail or a phone call from a terminal apparatus such as a smart phone. A notification of such information may be performed by the light emitting portion for notification, and may be performed by both of the vibration generation unit 175 and the light emitting portion.

The communication unit 250 performs a communication process with the external terminal apparatus 420 as described in FIG. 4. For example, a wireless communication process conforming to a standard such as Bluetooth (registered trademark) is performed. Specifically, the communication unit 250 performs a process of receiving a signal from the antenna 252 or a process of transmitting a signal to the antenna 252. A function of the communication unit 250 may be realized by a communication processor or a logic circuit such as an ASIC.

The biological information detection device or the like of the present embodiment may include a processor and a memory. The processor here may be a central processing unit (CPU). However, the processor is not limited to a CPU, and may be various processors such as a graphics processing unit (GPU) or a digital signal processor (DSP). The processor may be a hardware circuit using an application specific integrated circuit (ASIC). The memory stores a computer readable command, and each constituent element of the biological information detection device or the like according to the present embodiment is realized by the processor executing the command. The memory here may be a semiconductor memory such as an SRAM or a DRAM, and may be a register or a hard disk. The command here may be a command of a command set forming a program, and may be a command for instructing a hardware circuit of the processor to perform an operation.

2.3 Configuration Example of Sensor Unit

2.3.1 Entire Configuration Example of Sensor Unit

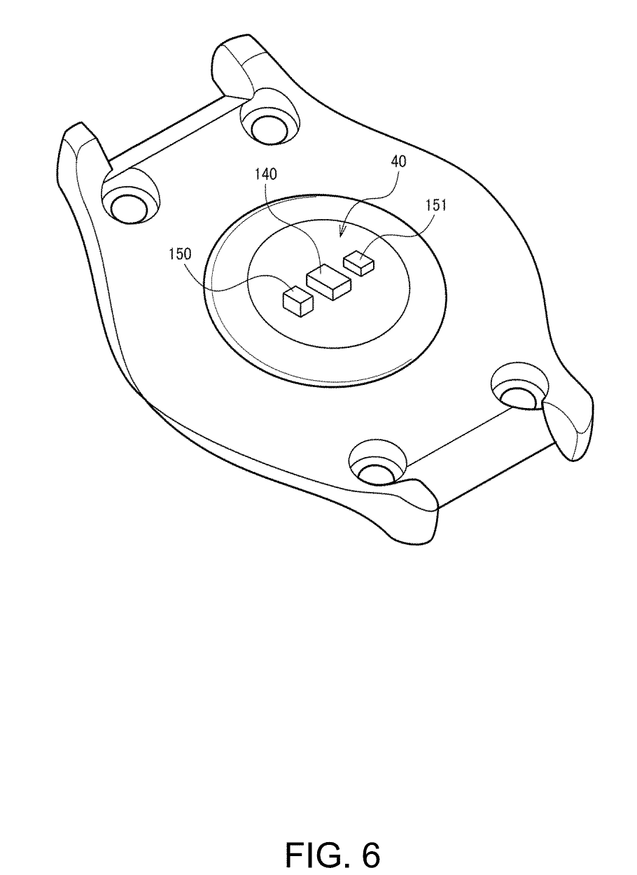

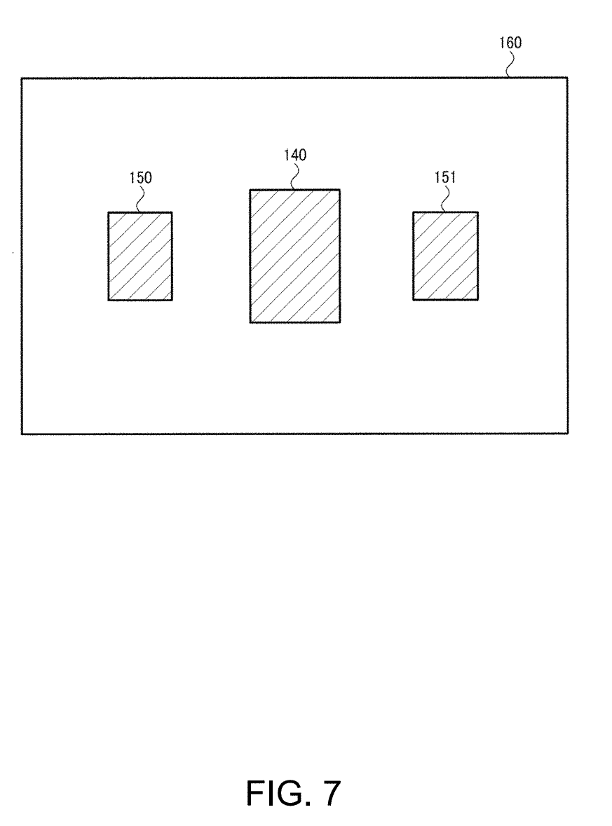

FIGS. 1(A) to 1(C), and FIGS. 6 and 7 illustrate a detailed configuration example of the sensor unit 40. FIG. 6 is a perspective view of the sensor unit 40, FIGS. 1(A) to 1(C) are sectional views of the sensor unit 40, and FIG. 7 is a plan view illustrating arrangement of the light receiving portion 140, and the first light emitting portion 150, and the second light emitting portion 151 on the board 160. FIG. 7 corresponds to a plan view in which the biological information detection device is viewed in a direction (an opposite direction to DR1 from a starting point which is set further toward DR1 side than the sensor unit 40) from a subject to the biological information detection device in a mounting state in FIG. 1(A) and the like.

The light receiving portion 140, the first light emitting portion 150, and the second light emitting portion 151 are mounted on the board 160 (sensor board). However, as will be described later, the first light emitting portion 150 may be mounted on the board 160 via other members. The light receiving portion 140 receives light (reflected light, transmitted light, or the like) from the subject. The first light emitting portion 150 and the second light emitting portion 151 emit light toward the subject. For example, the first light emitting portion 150 and the second light emitting portion 151 emit light toward the subject, and, if the light is reflected by the subject (blood vessel), the light receiving portion 140 receives and detects the reflected light.

The light receiving portion 140 may be implemented by, for example, a light receiving element such as a photodiode. The first light emitting portion 150 and the second light emitting portion 151 may be implemented by, for example, light emitting elements such as LEDs. The light receiving portion 140 may be implemented by, for example, a PN junction diode element formed on a semiconductor substrate. In this case, an angle limiting filter for narrowing a light reception angle or a wavelength limiting filter for limiting a wavelength of light which is incident to the light receiving element may be formed on the diode element.

For example, in a case of a pulsimeter, light from the light emitting portion travels through the inside of a subject and diffuses or scatters in the epidermis, the dermis, and the subcutaneous tissue. Then, the light reaches a blood vessel (detected part) and is reflected therefrom. At this time, some of the light is absorbed by the blood vessel. Since an absorption rate of the light in the blood vessel changes due to an influence of the pulse and thus an amount of the reflected light also changes, the light receiving portion 140 receives the reflected light so as to detect the change in the amount of light, thereby detecting a pulse rate which is biological information.

A light blocking member (light blocking wall) (not illustrated) may be provided between the first light emitting portion 150 or the second light emitting portion 151, and the light receiving portion 140. The light blocking member blocks light from, for example, the first light emitting portion 150 or the second light emitting portion 151 from being directly incident to the light receiving portion 140.

A diaphragm (not illustrated) may be provided in the sensor unit 40. The diaphragm restricts light from the subject or restricts light which is directly incident to the light receiving portion from the light emitting portion on an optical path between the subject and the sensor unit 40. The light blocking member 70 may be integrally formed with the diaphragm by performing sheet metal processing on a metal.

2.3.2 Relationship Between Pressure and Detection Signal

FIG. 8 is a diagram exemplifying a change of absorbance with respect to pressure. A transverse axis expresses pressure, and a longitudinal axis expresses absorbance. If pressure changes, a blood vessel influenced by the pressure changes. A blood vessel which is most easily influenced, that is, which is influenced by the lowest pressure is a capillary. In the example illustrated in FIG. 8, if the pressure exceeds p1, a change amount of the absorbance increases, and this indicates that a capillary starts to collapse due to the pressure. If the pressure exceeds p2, a change in the absorbance is smooth, and this indicates that the capillary almost completely collapses (closed). An artery is influenced next to a capillary. If the pressure further increases and exceeds p3, a change amount of the absorbance increases again, and this indicates that an artery starts to collapse due to the pressure. If the pressure exceeds p4, a change in the absorbance is smooth, and this indicates that the artery almost completely collapses (closed).

In the present embodiment, a signal corresponding to a capillary is detected as the second detection signal so that a ratio of body movement noise is increased, and a signal (pulse signal) corresponding to an artery is detected as the first detection signal so that a ratio of the pulse signal is increased. Thus, the pressure P2 at a contact position corresponding to the second light emitting portion 151 is designed to be included in the range of p1 to p2, and the pressure P1 at a contact position corresponding to the first light emitting portion 150 is designed to be included in the range of p3 to p4. A difference in the pressure between the first light emitting portion 150 and the second light emitting portion 151 is preferably, for example, between 2.0 kPa and 8.0 kPa.

FIG. 9 is a diagram exemplifying a change in body movement noise sensitivity with respect to pressure. FIG. 9 also illustrates examples in which a distance L between the light emitting portion and the light receiving portion is 2 mm and 6 mm. In either of the examples in which the distance L is 2 mm and 6 mm, there is a tendency that, as the pressure becomes lower, the noise sensitivity becomes higher, and as the pressure becomes higher, the noise sensitivity becomes lower. This may be because, since blood flowing through a capillary easily moves due to body movement, noise due to body movement is easily included in light reflected at a capillary which is present at a relatively shallow position in a biotissue.

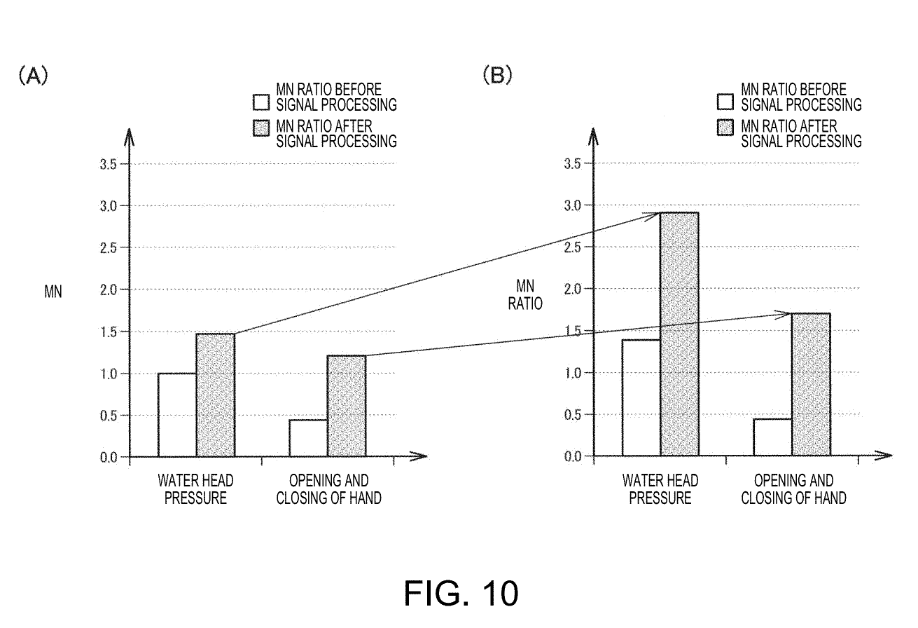

FIG. 10(A) illustrates a change in an MN ratio (SN ratio) of the first detection signal before and after the body movement noise reduction process is performed in a case where a pressure difference between the first light emitting portion 150 and the second light emitting portion 151 is not set, and only a difference between distances L1 and L2 to the light receiving portion 140 is set. Here, operations of changing water head pressure (a height relationship between the heart and a measurement part) and opening and closing the hand as movement of a user which causes body movement noise are performed, and the reduction extent of body movement noise corresponding to each operation is measured. Movement of changing the water head pressure is, for example, movement of changing a height of a measurement position, and may be realized by, for example, an action of raising or lowering the arm. Opening and closing of the hand may be realized by an action of alternately performing a state in which all the fingers are bent so as to close the hand and a state in which the fingers are stretched tightly so as to open the hand.

As can be seen from FIG. 10(A), it is possible to check an effect of reducing body movement noise by just setting a difference in a distance. In contrast, FIG. 10(B) illustrates a change in an MN ratio of the first detection signal before and after the body movement noise reduction process is performed in a case where a pressure difference between the first light emitting portion 150 and the second light emitting portion 151 is set, and a difference between distances L1 and L2 to the light receiving portion 140 is also set. As is clear from comparison between FIG. 10(A) and FIG. 10(B), it is possible to understand that a body movement noise reduction effect is improved by also setting a pressure difference. Therefore, a description will be made of an embodiment in which a pressure difference is mainly set.

In other words, when biological information of a subject is measured, in a case where pressure at a contact position with a subject in a position or a region corresponding to the first light emitting portion 150 is indicated by P1, and pressure at a contact position with the subject in a position or a region corresponding to the second light emitting portion 151 is indicated by P2, a relationship of P1>P2 is satisfied. In the above-described way, as described above, a difference can be set in characteristics of the first detection signal and the second detection signal.

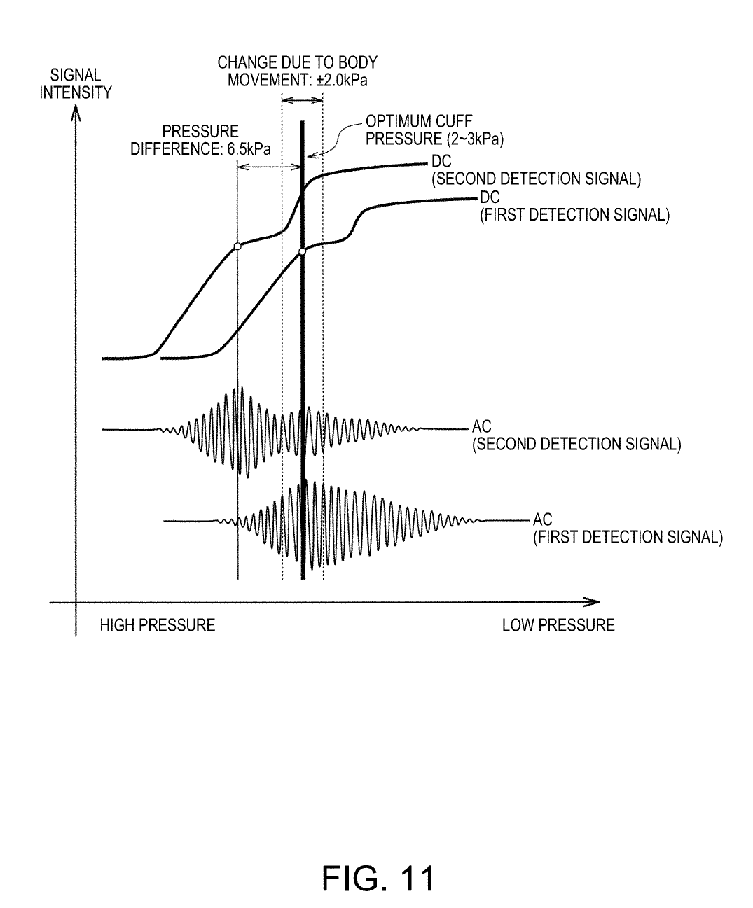

In FIG. 11, a transverse axis expresses cuff pressure (in a case of the biological information detection device 400 illustrated in FIG. 2(A), pressure caused by the band 10), and a longitudinal axis expresses a DC component and an AC component of a detection signal. As can be seen from DC signals illustrated in an upper part in FIG. 11, in the first detection signal in which pressure is relatively high, some pressure is applied even in a state in which cuff pressure is relatively low, and a DC component is restricted. In contrast, since pressure in the second detection signal is relatively low, the extent of a DC component being restricted is lower than in the first detection signal in a state of predetermined cuff pressure. Thus, in a range of “optimum cuff pressure” illustrated in FIG. 11, pressure corresponding to the first light emitting portion 150 is included in the range of p3 to p4, and thus noise is restricted so that a level of a pulse signal increases. On the other hand, pressure corresponding to the second light emitting portion 151 is included in the range of p1 to p2, and thus noise is insufficiently restricted so that a ratio of body movement noise increases.

This is also clear from comparison between AC components illustrated in a lower part in FIG. 11, and, in the range of the optimum cuff pressure, the first detection signal has a high level of an AC component, and the second detection signal has a low level of the AC component. As described above, since a pulse signal indicates a change in a detection signal, that is, appears in an AC component, FIG. 11 illustrates that whereas the pulse signal is sufficiently detected in the first detection signal, a ratio of body movement noise is relatively high in the second detection signal.

2.3.3 Height of Contact Position with Subject in Position or Region Corresponding to Light Emitting Portion

A pressure difference may be realized, specifically, due to a difference between heights at positions of coming into contact with a subject. As described above, pressure is increased at a contact position corresponding to the first light emitting portion 150 for mainly detecting a pulse signal, and pressure is lower than in the first light emitting portion 150 at a contact position corresponding to the second light emitting portion 151.

This is because, since the biological information detection device protrudes toward a subject side as a height is increased, when the biological information detection device 400 is fixed on the wrist or the like under predetermined cuff pressure, pressure corresponding to the first light emitting portion 150 with a large height can be made higher than pressure corresponding to the second light emitting portion 151 having a small height. This is illustrated in FIG. 11 described above. Hereinafter, a specific structure for setting a height difference will be described.

First, the above-described H1, that is, “a height of a contact position or a contact region with a subject in a position or a region corresponding to the first light emitting portion 150” maybe a height of a contact position or a contact region with a subject in a region where the first light emitting portion 150 is disposed. Similarly, H2 may be a height of a contact position or a contact region with a subject in a region where the second light emitting portion 151 is disposed. In other words, the position or the region corresponding to the first light emitting portion 150 (second light emitting portion 151) maybe an arrangement region for the first light emitting portion 150 (second light emitting portion 151).

Here, the arrangement region is a region where an element is disposed in the sensor unit 40, and may be a region of the element itself in a plan view viewed from a direction orthogonal to the board 160 in a case where the element is mounted on the board 160 (or a height adjustment member provided on the board 160) as illustrated in FIG. 1(A). The “element” here may indicate only an LED, but is not limited thereto, and may indicate the entire package including the LED. For example, a set of an LED, a light irradiation lens, and a sealing resin is formed as a package, the entire package is recognized as the “element” (for example, the first light emitting portion 150). This is also the same for the light receiving portion 140, and only a PD formed through PN junction is not recognized as the light receiving portion 140, but may also be recognized as the light receiving portion 140 including an optical filter and the like formed as a single package.

There may be various heights of a contact position with a subject in the arrangement region. If the first light emitting portion 150 comes into contact with a subject as a single point in the arrangement region, the point is a contact position with the subject in the arrangement region of the first light emitting portion 150. However, as described above, since some pressure is required to be applied to a subject in measurement of pulse wave information, a contact position is expected to be a certain extent of region (in a narrow sense, the entire surface of the first light emitting portion 150 on the subject side) in the arrangement region of the first light emitting portion 150. In this case, a contact position with the subject may be any point in the arrangement region, may be a region having a certain extent of area, and may be the entire arrangement region. In a case where a contact position is a region, a height may differ depending on a position. In this case, an average height or the like maybe used as a height at the contact position.

As an example of this case, there may be a structure in which each element of the first light emitting portion 150 and the second light emitting portion 151 comes into contact with a subject. Specifically, in a case where a height to the surface of the first light emitting portion 150 on the subject side is indicated by HA1, and a height to the surface of the second light emitting portion 151 on the subject side is indicated by HA2, a relationship of HA1>HA2 is satisfied, and thus a relationship of H1>H2 maybe satisfied. In other words, a height of each light emitting portion at a contact position maybe a height of the surface of each of the first light emitting portion 150 and the second light emitting portion 151 on the subject side.

For example, as illustrated in FIG. 6 and the like, in a case where each of the first light emitting portion 150 and the second light emitting portion 151 has a rectangular parallelepiped shape, a height of one surface on a subject side among six surfaces of the rectangular parallelepiped is a height at a contact position.

In the example illustrated in FIG. 6 and the like, since it is assumed that the surface of the first light emitting portion 150 on the subject side is parallel to a predetermined reference surface, a height in a contact region is uniform in the entire region. However, in a case where the surface of the first light emitting portion 150 on the subject side is not parallel to the predetermined reference surface, a height with respect to the reference surface changes depending on a position in the contact region. Therefore, in the present embodiment, for example, a height of a contact region with a subject may be an average height of heights at respective points included in the contact region. In the above-described way, also in a case where a contact region having a certain extent of area is a target, a height thereof can be appropriately defined.

More specifically, as illustrated in FIGS. 1(A) and 1(B), the second light emitting portion 151 and the light receiving portion 140 are provided on the board 160, and a height adjustment member (161 or 162) maybe provided between the first light emitting portion 150 and the board 160. In FIGS. 1(A) to 1(C), a resin 60 is used to fix the first light emitting portion 150, the second light emitting portion 151, and the light receiving portion 140. In FIGS. 1(A) to 1(C), the resin 60 does not contribute to a height at a contact position.

In the above-described way, by using the height adjustment member, it is possible to set a difference between a height of the first light emitting portion 150 in the arrangement region and a height of the second light emitting portion 151 in the arrangement region. In this case, since a height difference is adjusted by the height adjustment member, it is possible to loosen restrictions in a height (which is a length of the first light emitting portion 150 in the direction DR1 and is LH1 which will be described later) of the first light emitting portion 150 and a height (LH2) of the second light emitting portion 151. For example, since light emitting portions having the same structure (LH1=LH2) can be used as the first light emitting portion 150 and the second light emitting portion 151, it is possible to alleviate a load to supply or adjust elements. Alternatively, regarding a height of an element, even in a case where a height of the second light emitting portion 151 is larger (LH1<LH2) for some reason, a height of the first light emitting portion 150 with respect to the predetermined reference surface can be made larger (HA1>HA2) by using a height adjustment member with a sufficient height.

Here, as illustrated in FIG. 1(A), the height adjustment member may be a second board 161. In this case, an external connection terminal of the first light emitting portion 150 is connected to a connection terminal provided on the board 160 via a through hole of the second board 161.

In a case of the configuration illustrated in FIG. 1(A) and the like, the supply of power to each element or input and output of signals are expected to be performed through the board 160 (main board). In other words, even if a height adjustment member is provided, the first light emitting portion 150 has to be electrically connected to the board 160. In relation to this fact, in a case where the second board 161 is used as a height adjustment member, the height adjustment member includes wirings, and thus it is easy to connect the first light emitting portion 150 and the board 160 to each other. Specifically, through holes of the second board 161 may be used.

However, electrical connection between the first light emitting portion 150 and the board 160 is not limited thereto, and, as illustrated in FIG. 1(B), an external connection terminal of the first light emitting portion 150 may be connected to a connection terminal provided on the board 160 via a wire WI.

In this case, since connection between the first light emitting portion 150 and the board 160 is realized by using the wire WI, a height adjustment member is not required to have a structure for electrical connection, and may be implemented by, for example, an insulator.

A height of the light emitting portion 150 may be set to differ without providing a height adjustment member. Specifically, as illustrated in FIG. 1(C), in a case where a length of the first light emitting portion 150 in the first direction DR1 is indicated by LHB1, and a length of the second light emitting portion 151 in the first direction DR1 is indicated by LH2, a relationship of LHB1>LH2 is satisfied, and thus a relationship of H1>H2 may be satisfied. Here, DR1 indicates a direction directed from the sensor unit 40 toward a subject when biological information is detected as illustrated in FIG. 1(A). Here, the direction DR1 is a direction directed toward the subject side perpendicularly to the board 160. Alternatively, the direction DR1 is a direction directed toward the subject perpendicularly to an upper surface of the first light emitting portion 150, an upper surface of the second light emitting portion 151, or an upper surface of the light receiving portion 140. A height difference may be set between the first light emitting portion 150 and the second light emitting portion 151 by changing a height of a resin sealed case of the light emitting portion 150.

In the above-described way, a height adjustment member is not required to be provided unlike in the case illustrated in FIGS. 1(A) or 1B, and thus it is possible to reduce the number of components.

The sensor unit of the present embodiment is not limited to a structure in which each element of the first light emitting portion 150 and the second light emitting portion 151 comes into contact with a subject. For example, as illustrated in FIG. 12(A), the sensor unit 40 may include the light transmissive member 50 which is provided at a position located further toward a subject side than the first light emitting portion 150, transmits light from the subject therethrough, and comes into contact with the subject so as to apply pressure thereto when biological information of the subject is measured. In this case, in a case where a height of the light transmissive member 50 in a position or a region corresponding to the first light emitting portion 150 is indicated by HD1, a relationship of HD1>H2 is satisfied, and thus a relationship of H1>H2 is satisfied. In other words, a height in a position or a region corresponding to the first light emitting portion 150 may be a height of the light transmissive member 50 in the position or the region corresponding to the first light emitting portion 150.

Here, H2 may be, specifically, a height HA2 to a surface of the second light emitting portion 151 on the subject side, may be a length LH2 of the second light emitting portion 151 in the direction DR1, and may be a height HD2 of the light transmissive member 50 provided on the subject side of the second light emitting portion 151.

The light transmissive member 50 is provided on a surface of the biological information detection device 400 on a side coming into contact with a subject, and transmits light from the subject therethrough. The light transmissive member 50 comes into contact with a subject when biological information of the subject is measured. For example, as illustrated in FIG. 12(A), a protrusion 52 of the light transmissive member 50 may come into contact with the subject. A surface shape of the protrusion 52 is preferably a curved shape (spherical shape), but is not limited thereto, and may employ various shapes. The light transmissive member 50 may be optically transparent to a wavelength of light from the subject, may be formed by using a transparent material, and may be formed by using a colored material. By using a colored material, the light transmissive member 50 may function as a band-pass filter which blocks light other than light with a detection target wavelength band.

There may be the occurrence of various modifications of a method of defining a height of the light transmissive member 50, and, for example, a distance from a predetermined reference surface (for example, in the same manner as in the above-described example, the surface of the board 160) may be used as a height.

Since a height difference is preferably set, a shape of the light transmissive member 50 may be variously modified as illustrated in FIGS. 12B and 12C. For example, the light transmissive member 50 may be provided not only in a position or a region corresponding to the first light emitting portion 150 but also in a position or a region corresponding to the second light emitting portion 151. Specifically, as illustrated in FIG. 12(C), the light transmissive member 50 maybe provided at a position located further toward a subject side than the second light emitting portion 151, transmits light from the subject therethrough, and comes into contact with the subject so as to apply pressure thereto when biological information of the subject is measured. In a case where a height of the light transmissive member 50 in a position or a region corresponding to the second light emitting portion 151 is indicated by HD2, a relationship of HD1>HD2 is satisfied, and thus a relationship of H1>H2 may be satisfied.

In this case, a plurality of (for example, a number corresponding to the number of photoelectric sensors) protrusions 52 may be provided. In the example illustrated in FIG. 12(C), a protrusion 52-1 is provided for a first photoelectric sensor formed of the first light emitting portion 150 and the light receiving portion 140, and a protrusion 52-2 is provided for a second photoelectric sensor formed of the second light emitting portion 151 and the light receiving portion 140.

There may be various methods of defining a position or a region corresponding to each light emitting portion. For example, the height HD1 may be a height of the light transmissive member 50 at a representative position of the first light emitting portion 150, and the height HD2 may be a height of the light transmissive member 50 at a representative position of the second light emitting portion 151. Here, for example, a central position and the like of each light emitting portion may be used as the representative position here.

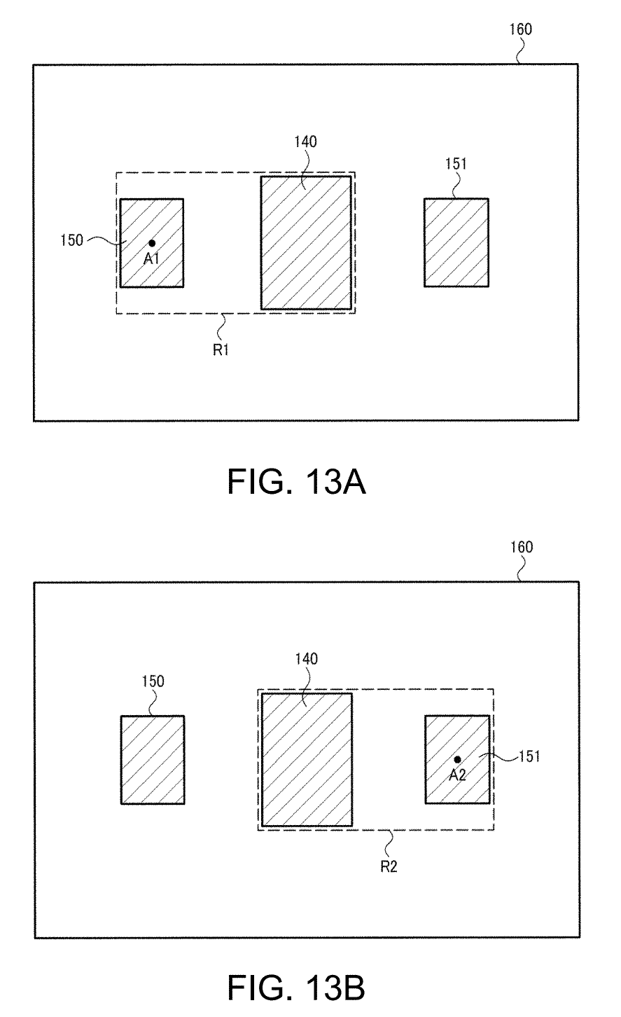

In this case, a central position of the first light emitting portion 150 is indicated by A1 in FIG. 13(A), and a central position of the second light emitting portion 151 is indicated by A2 in FIG. 13(B). An intersection between a straight line extending from A1 in the direction DR1 (as illustrated in FIG. 12(A), a direction directed toward a subject side perpendicularly to the board 160) and a surface (a surface coming into contact with a subject when mounted) of the light transmissive member 50 is defined, and the height HD1 of the light transmissive member 50 at the intersection may be used as a height of the light transmissive member 50 at the central position Al of the first light emitting portion 150. This is also the same for the height HD2 of the light transmissive member 50 at the central position A2 of the second light emitting portion 151.

Alternatively, in a case where a region including the first light emitting portion 150 and the light receiving portion 140 is referred to as a first region, and a region including the second light emitting portion 151 and the light receiving portion 140 is referred to as a second region, in a plan view viewed from a subject side, the height HD1 may be an average height of the light transmissive member 50 in the first region, and the height HD2 may be an average height of the light transmissive member 50 in the second region.

Here, the plan view viewed from the subject side corresponds to a state in which the biological information detection device is observed in a direction of DR2 (an opposite direction to DR1 as illustrated in FIG. 12(A)) from a point of sight which is set further toward the subject side (DR1 side) than the first light emitting portion 150 or the like in FIG. 12(A), and corresponds to, specifically, states illustrated in FIGS. 13(A) and 13(B). There may be various regions including the light emitting portion and the light receiving portion, and, as an example, there maybe a rectangular region which includes the light emitting portion and the light receiving portion and whose area is the minimum. In this case, a region (first region) corresponding to the first light emitting portion 150 is R1 in FIG. 13(A), and a region (second region) corresponding to the second light emitting portion 151 is R2 in FIG. 13(B).

When an intersection between straight lines extending in the direction DR1 from respective points included in the region R1 and a surface of the light transmissive member 50 are defined, a height of the light transmissive member in the region corresponding to the first light emitting portion 150 maybe obtained by averaging heights of the light transmissive member 50 at the intersections. For example, an average value of heights of the light transmissive member 50 in the range illustrated in FIG. 12(A) is HD1. FIG. 12(A) illustrates only a single section, but heights may also be averaged in a height direction in FIG. 12(A). In the same manner for the second light emitting portion 151, an average height in the region R2 may be HD2.

A shape of the light transmissive member 50 in a position or a region corresponding to the light receiving portion 140 may be variously modified. In FIG. 12(A) or 12C, the light transmissive member 50 is provided on the light receiving portion 140 on the subject side. On the other hand, in FIG. 12(B), the light transmissive member 50 is not provided on the light receiving portion 140 on the subject side.

The first light emitting portion 150, or members other than the light transmissive member 50 may come into contact with a subject. For example, the sensor unit 40 may include a first member 181 provided between at least the first light emitting portion 150 and the light receiving portion 140 and a second member 182 provided between at least the second light emitting portion 151 and the light receiving portion 140. In a case where a height of the first member 181 is indicated by HC1, and a height of the second member 182 is indicated by HC2, a relationship of HC1>HC2 is satisfied, and thus a relationship of H1>H2 is satisfied. In other words, a height in a position or a region corresponding to the first light emitting portion 150 may be a height of the first member 181, and a height in a position or a region corresponding to the second light emitting portion 151 may be a height of the second member 182.

The first member 181 and the second member 182 here may have structures for realizing a pressure difference and may stabilize a contact state between the sensor unit 40 and a subject. The first member 181 and the second member 182 may be two different members, but are not limited thereto, and may be variously modified.

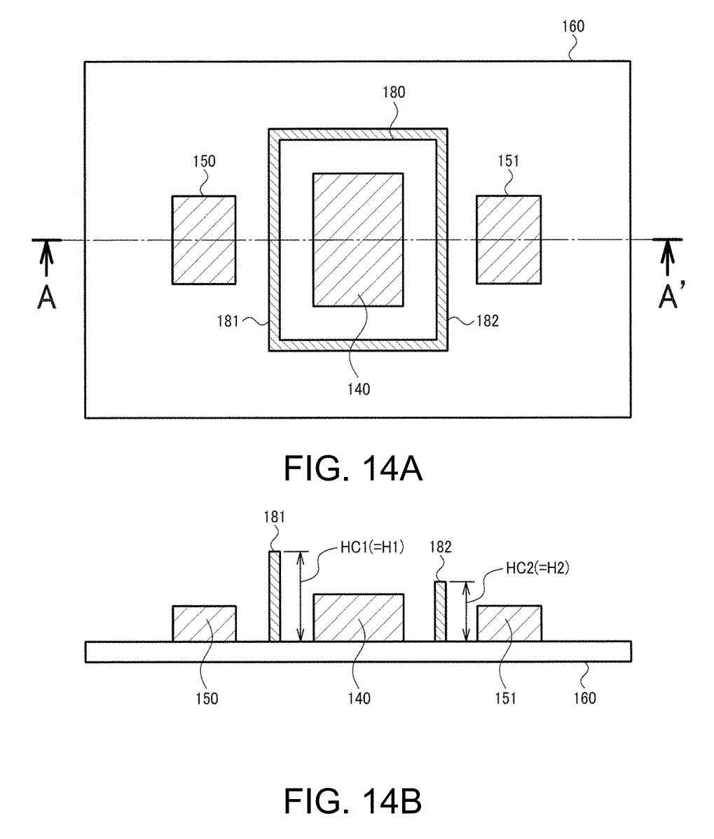

For example, the first member 181 and the second member 182 may be parts of a frame portion 180. The frame portion 180 is provided around the light receiving portion 140 as illustrated in FIG. 14(A). FIG. 14(A) is a plan view from a direction perpendicular to the board 160 in the same manner as FIG. 7 or the like. In the example illustrated in FIG. 14(A), frame portion 180 surrounding the light receiving portion 140 has a square shape, but is not limited thereto, and may have a circular shape or other polygonal shapes. Alternatively, the frame portion 180 is not required to be formed in a continuous shape, and the frame portion 180 (which is formed of a plurality of circular arcs which are not in contact with each other) having a gap may be used.

In a case of FIG. 14(A), the first member 181 corresponds to one side of the frame portion 180 on the first light emitting portion 150 side, and the second member 182 corresponds to one side of the frame portion 180 on the second light emitting portion 151 side. FIG. 14(B) illustrates heights of the first member 181 and the second member 182 in this case. FIG. 14(B) is a sectional view which is viewed from a direction along the board in the same manner as FIG. 1(A) or the like, and, specifically, is a sectional view taken along the line A-A′ in FIG. 14(A).

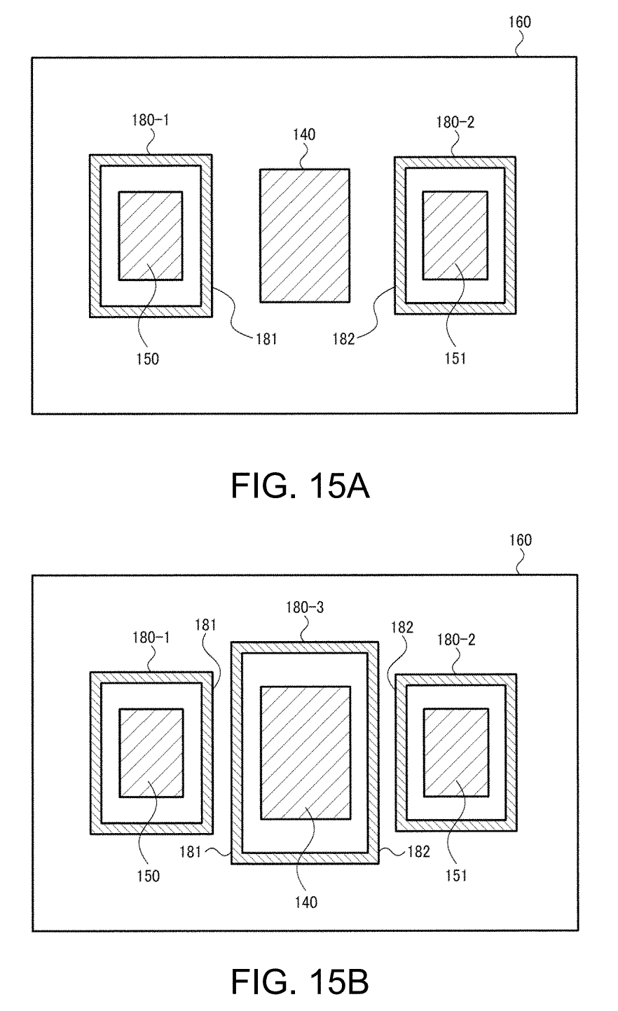

As illustrated in FIG. 15(A), the frame portion 180 may be formed of a first frame portion 180-1 provided around the first light emitting portion 150, and a second frame portion 180-2 provided around the second light emitting portion 151. In this case, the first member 181 corresponds to one side of the first frame portion 180-1 on the light receiving portion 140 side, and the second member 182 corresponds to one side of the second frame portion 180-2 on the light receiving portion 140 side.

Alternatively, as illustrated in FIG. 15(B), the frame portion 180 may be formed of a first frame portion 180-1 provided around the first light emitting portion 150, a second frame portion 180-2 provided around the second light emitting portion 151, and a third frame portion 180-3 provided around the light receiving portion 140. In this case, the first member 181 corresponds to at least one of one side of the first frame portion 180-1 on the light receiving portion 140 side and one side of the third frame portion 180-3 on the first light emitting portion 150 side. The second member 182 corresponds to at least one of one side of the second frame portion 180-2 on the light receiving portion 140 side and one side of the third frame portion 180-3 on the second light emitting portion 151 side.

In a case where a height of a contact position with a subject in a position or a region corresponding to the light receiving portion 140 is indicated by H3, a relationship of H1≧H3≧H2 (here, a case of H1=H3=H2 is excluded) may be satisfied.

A height of a contact position with a subject in a position or a region corresponding to the light receiving portion 140 maybe the same as a height of a contact position with a subject in a position or a region corresponding to the first light emitting portion 150. In FIGS. 1(A) to 1(C) or FIG. 12(B), the height is a height of the light receiving portion 140 in the arrangement region, and, in FIGS. 12(A) and 12(C), the height is an average height or the like of the light transmissive member 50.

As illustrated in FIG. 7 or the like, in the present embodiment, there is a case where the light receiving portion 140 is provided between the first light emitting portion 150 and the second light emitting portion 151. In this case, if the light receiving portion 140 is extremely higher than the first light emitting portion 150 and the second light emitting portion 151 (H3>H1>H2), pressure corresponding to the first detection signal is mainly caused by the height of the light receiving portion 140, and pressure corresponding to the second detection signal is also mainly caused by the height of the light receiving portion 140. In other words, even if a difference is set between heights of the first light emitting portion 150 and the second light emitting portion 151, a pressure difference between the first detection signal and the second detection signal is reduced, and thus there is concern that processing accuracy may deteriorate. Therefore, also in order to clarify a pressure difference, a height relationship including the light receiving portion 140 may be defined, and, for example, a relationship of H1≧H3≧H2 (here, a case of H1=H3=H2 is excluded) may be satisfied. There may be the occurrence of a modification such as a relationship of H1>H2>H3, but the intensity of reflected light from a subject is weakened, and thus a distance between the subject and the light receiving portion 140 is preferably short. In other words, H3 is preferably a certain extent of size, and the relationship of H1≧H3≧H2 is also obtained by taking into consideration this fact.

By using the above-described configuration, the light receiving portion 140 receives light from a subject in a case where pressure applied to a measurement part of the subject is the first pressure, and light from the subject in a case where the pressure applied to the measurement part of the subject is the second pressure P2 lower than the first pressure. Consequently, it is possible to appropriately reduce noise (body movement noise) included in a detection signal and thus to perform a process of calculating highly accurate pulse wave information.

2.3.4 Distance Between Light Emitting Portion and Light Receiving Portion

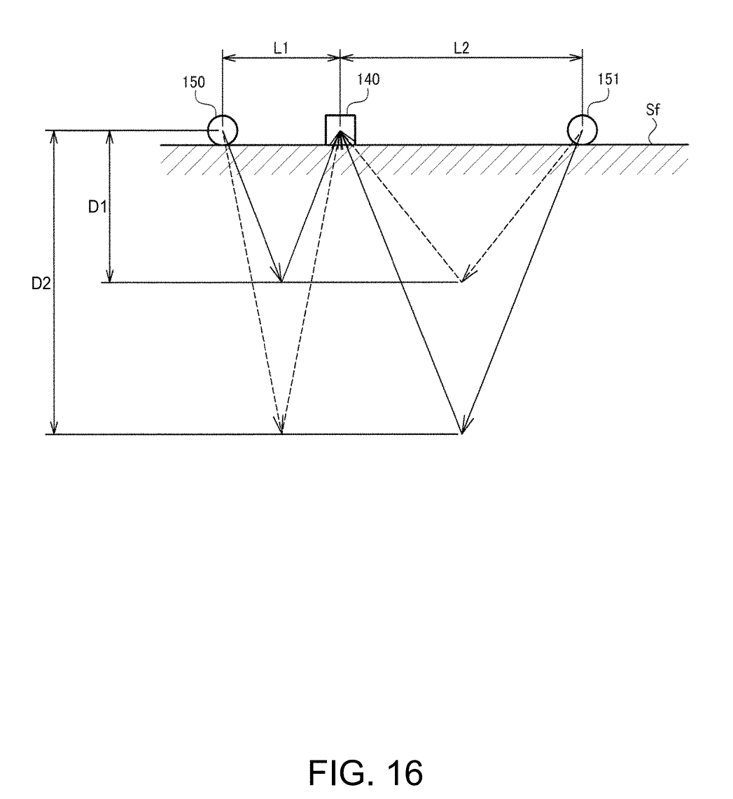

Next, a description will be made of the distance L1 between the first light emitting portion 150 and the light receiving portion 140 and the distance L2 between the second light emitting portion 151 and the light receiving portion 140. FIG. 16 is a diagram for explaining the influence which a distance between the light emitting portion and the light receiving portion exerts on a permeation depth of light. The first light emitting portion 150 and the light receiving portion 140, and the second light emitting portion 151 and the light receiving portion 140 come into contact with a skin surface Sf of a user's wrist. Here, as described above, the light receiving portion 140 is shared by the two light emitting portions. In the embodiment illustrated in FIGS. 12(A) to 12(C), actually, the light transmissive member 50 comes into contact with a subject, but, in FIG. 16, for simplification of description, the light transmissive member 50 is not illustrated.

It is known that, as a distance between the light emitting portion and the light receiving portion becomes shorter, the sensitivity for a deep part in a living body becomes lower than the sensitivity for a shallow part. In other words, with reference to light applied from the first light emitting portion 150, the intensity of light which is reflected at a position of the depth D1 in a biotissue and reaches the light receiving portion 140 is higher than the intensity of light which is reflected at a position of the depth D2 deeper than the depth D1 and reaches the light receiving portion 140. On the other hand, with respect to light applied from the second light emitting portion 151, the intensity of light which is reflected at a position of the depth D1 in a biotissue and reaches the light receiving portion 140 is higher than the intensity of light which is reflected at a position of the depth D2 deeper than the depth D1 and reaches the light receiving portion 140, but there is no occurrence of a difference from the first light emitting portion 150. Thus, the first light emitting portion 150 is suitable for measurement of a pulse wave of a blood vessel which is located at a shallower position than the second light emitting portion 151.

FIG. 17 is a diagram illustrating a relationship between a distance LD between the light emitting portion and the light receiving portion 140, and the signal intensity. The distance LD between the light emitting portion and the light receiving portion 140 is a distance between, for example, central positions (representative positions) of the light emitting portion and the light receiving portion. For example, in a case where the light receiving portion 140 has a rectangular shape (substantially a rectangular shape), a position of the light receiving portion 140 is a central position of the rectangular shape. In a case where the light emitting portion has a lens (not illustrated), a position of the light emitting portion 150 is, for example, a central position (a position of an LED chip) of the lens.

As is clear from FIG. 17, as the distance LD between the light emitting portion and the light receiving portion 140 becomes shorter, the intensity of a detection signal increases, and thus detection performance such as sensitivity improves. Therefore, with respect to the first light emitting portion 150 for first detection, which mainly detects a pulse signal, the distance LD to the light receiving portion 140 becomes preferably shorter.

In this case, as illustrated in FIG. 17, the distance between the light receiving portion 140 and the first light emitting portion 150 preferably satisfies a relationship of LD<3 mm. For example, as is clear from a tangential line G2 on a long distance side in a characteristic curve G1 in FIG. 17, the characteristic curve G1 saturates in a range of LD 3 mm. In contrast, in the range of LD<3 mm, the signal intensity considerably increases as the distance LD becomes shorter. Therefore, in this sense, a relationship of LD<3 mm is preferably satisfied. For example, the distance L1 between the first light emitting portion 150 and the light receiving portion 140 uses L1=about 1.0 to 3.0 mm.

The distance LD has a lower limit value, and it is not preferable that the distance LD is too short. FIG. 18 is a schematic diagram illustrating a state in which light emitted from the light emitting portion is reflected and scattered in a living body, and some light is received by the light receiving portion. In this case, light from the light emitting portion diffuses or scatters toward a blood vessel or the like of a subject, and the light is incident to the light receiving portion 140 so that a pulse wave is detected. In FIG. 18, the distance LD between the light emitting portion and the light receiving portion 140, and a measurement distance LB in a depth direction generally have a relationship of LD=2×LB. For example, a measurement limit distance in an optical detection unit formed of the light emitting portion and the light receiving portion 140 separated by the distance LD is about LB=LD/2. There is no blood vessel as a detection target object using a pulse wave in a range in which the distance LB is, for example, 100 μm to 150 μm. Therefore, if the distance LD is LD≦2×LB=2×100 μm to 2×150 μm=0.2 mm to 0.3 mm, it is expected that a pulse wave detection signal is considerably reduced. In other words, if the distance LD is short, the measurement distance LB in the depth direction is thus reduced. Therefore, if there is no detection target object in a range of the measurement distance LB, a detection signal is considerably reduced. In other words, as the distance LD becomes shorter, detection performance improves, but there is limit therein, and thus a lower limit value is present. In the present embodiment, since it is necessary to detect a pulse signal with sufficient intensity on the basis of light from the first light emitting portion 150, a relationship of L1≧1.0 mm is approximately set. In other words, preferably, a relationship of 1.0 mm≦L1≦3.0 mm is satisfied.

In contrast, the distance L2 between the second light emitting portion 151 and the light receiving portion 140 may be set so that the sensitivity for a pulse signal is lower and the sensitivity for body movement noise is higher than in the first light emitting portion 150. For example, if L2<1.0 mm or 3.0 mm<L2, a level of a pulse signal is reduced, and a level of body movement noise increases (an MN ratio is reduced) compared with the first light emitting portion 150 in which a relationship of 1.0 mm≦L1≦3.0 mm is satisfied.

However, an MN ratio (here, M indicates a pulse signal, N indicates noise, and the MN ratio is a ratio (general SN ratio) between the pulse signal and the noise) of a detection signal based on light from the second light emitting portion 151 is preferably sufficiently lower than an MN ratio of a detection signal based on light from the first light emitting portion 150. In other words, importance maybe placed on the fact that a value of L2 with respect to L1 is changed so that some difference occurs between the first and second detection signals (for example, to the extent to which a noise reduction process can be performed according to a spectrum subtraction method which will be described later), rather than the fact that a distance is set as an average value such as L2<1.0 mm or 3.0 mm<L2.

In other words, in the second detection signal based on light from the second light emitting portion 151, since an MN ratio has only to be sufficiently lower than that in the first detection signal, a pulse component may be included to some degree, that is, L2 may satisfy a relationship of 1.0 mm≦L2≦3.0 mm.

Here, a relationship between L1 and L2 for causing a difference between the first and second detection signals may be, for example, L2>2×L1 or the like. In this case, since L2>2.0 mm at L1=1.0 mm, L2 may be 2.5 mm or the like, and a pulse signal is detected to a certain level of intensity, but the condition can be satisfied in which an MN ratio of the second detection signal is lower than that of the first detection signal in which the shorter distance L1 is set.

If body movement noise is relatively increased in the second detection signal, as described above, the distance L2 maybe a considerably small value. In other words, a distance between the light receiving portion 140 and each light emitting portion may be determined according to a relationship of L2<L1, for example, L2<L1/2. However, when taking into consideration a case where a light blocking wall is provided in order to block light from being directly incident to the light receiving portion 140 from each light emitting portion, it may be difficult for the distance L1 or L2 to be set to an extremely small value. For example, if L1=1.0 mm, a relationship of L2<0.5 mm is required to be satisfied, and thus it may be hard to dispose each component due to a space. In a case of taking into consideration this fact, a relationship of L2>L1 is preferably satisfied, and a relationship of L2<L1 may be employed depending on a situation.

As a specific numerical value, L1 may satisfy a relationship of 1 mm≦L1≦3 mm, and L2 may satisfy a relationship of 2 mm≦L2. However, as described above, if importance is placed on a relative relationship with L1, a condition regarding L2 is required to satisfy not only a relationship of 2 mm≦L2 but also the relative relationship. As an example, the relative relationship is L2>L1, and L2≧2 mm. Alternatively, under a more strict condition, a relationship of L2>2×L1 and L2≧2 mm may be satisfied.

2.3.5 Modification Examples of Arrangement of Light Emitting Portion and Light Receiving Portion

Next, a description will be made of modification examples of arrangement of the light emitting portions and the light receiving portion on the board 160. In FIGS. 1(A) to 1(C) and the like, the light receiving portion 140 is disposed between the first light emitting portion 150 and the second light emitting portion 151.

However, arrangement of the light receiving portion 140 and a plurality of light emitting portions is not limited thereto. For example, as illustrated in FIG. 19(A), the light receiving portion 140, the first light emitting portion 150, and the second light emitting portion 151 may be arranged in this order and be mounted in a predetermined direction.

In this case, a difference naturally occurs between the light emitting portions and the light receiving portion, and, specifically, a relationship of L1<L2 is satisfied as illustrated in FIG. 19(A). In a narrower sense, a relationship of L2>2×L1 may be satisfied.

Also in a case of the arrangement illustrated in FIG. 19(A), the height H1 of a contact position with a subject in a position or a region corresponding to the first light emitting portion 150 may be larger than the height H2 of a contact position with the subject in a position or a region corresponding to the second light emitting portion 151. As a structure for setting a height difference, any one of the structures described in FIGS. 1(A) to 1(C) and the like may be employed.

In the arrangement (hereinafter, a plurality of light emitting portions oppose each other with respect to the light receiving portion 140, and thus the arrangement will be referred to as opposing arrangement in some cases) illustrated in FIG. 7, an optical path from the first light emitting portion 150 to the light receiving portion 140 does not overlap an optical path from the second light emitting portion 151 to the light receiving portion 140. Thus, in a case of an embodiment in which the light transmissive member 50 is provided as illustrated in FIG. 12(C) or the like, the protrusion 52-1 and the protrusion 52-2 hardly interfere with each other, and there is an advantage in that a difference between the heights HD1 and HD2 can be easily set.

Specifically, as can be seen from comparison between FIGS. 13(A) and 13(B), and FIG. 19(B), in the opposing arrangement, an overlapping portion between the region R1 corresponding to the first light emitting portion 150 and the region R2 corresponding to the second light emitting portion 151 is smaller than in the arrangement illustrated in FIG. 19(A). Thus, even when an average of heights is obtained, an overlapping portion is smaller and a height difference is more easily set than in the example illustrated in FIGS. 19(A) and 19(B).

On the other hand, in a case of the modification example illustrated in FIG. 19(A), optical paths overlap each other, and thus there is an advantage in that a correlation level between the first detection signal and the second detection signal can be heightened. As described above, in order to increase an effect of a noise reduction process, detection signals preferably have a certain extent of correlation and have different characteristics. In other words, if importance is placed on correlation between the first and second detection signals, it is advantage to use the arrangement described with reference to FIG. 19(A).

In the above description, a description has been made of an example in which two photoelectric sensors, that is, at least one light receiving portion and two light emitting portions are included in the biological information detection device 400, but any other configuration may be used, and the biological information detection device may include three or more photoelectric sensors. In this case, a single light receiving portion may be shared by all light emitting portions, a light receiving portion forming a set with each light emitting portion may be provided, and a combination thereof may be used. In other words, the biological information detection device may include first to N-th (where N is an integer of 3 or more) light emitting portions and first to k-th (where k is an integer satisfying 1≦k≦N) light receiving portions.

3. Timing Control

As described above, in the present embodiment, it is assumed that a single light receiving portion 140 is shared by a plurality of light emitting portions. In this case, if light beams from the plurality of light emitting portions are simultaneously incident to the light receiving portion 140, it is hard for the light receiving portion 140 to separate and process the light beams. As a result, even if two signals having different characteristics are to be acquired by setting a pressure difference, it is not possible to perform an appropriate process since the signals are mixed with each other.

Therefore, in the present embodiment, the processing unit 200 performs a biological information detection process on the basis of a first light reception result (first detection signal) in the light receiving portion 140 at a first timing and a second light reception result (second detection signal) in the light receiving portion 140 at a second timing which is different from the first timing.

In the above-described way, it is possible to acquire the first detection signal in a case where pressure is the first pressure P1 and the second detection signal in a case where pressure is the second pressure P2 at different timings (the signals are acquired at exclusive timings). In other words, it is possible to prevent the first detection signal from being mixed with the second detection signal in the light receiving portion 140.

This process can be performed by appropriately controlling light emission timings in the first light emitting portion 150 and the second light emitting portion 151 and a light reception timing in the light receiving portion 140. In other words, as described above, the biological information detection device 400 (sensor unit 40) according to the present embodiment includes the first light emitting portion 150 and the second light emitting portion 151 as light emitting portions, and the processing unit 200 performs a biological information detection process on the basis of the first detection signal in the light receiving portion 140 at the first timing based on light emission from the first light emitting portion 150 and the second detection signal in the light receiving portion 140 at the second timing based on light emission from the second light emitting portion 151.

It is not necessary to process the first detection signal and the second detection signal at different timings with respect to digital signals obtained by A/D-converting the detection signals. In other words, it is noted to focus on not an acquisition timing of a detection signal in the processing unit 200 but a light reception timing (or light emission timings in the first light emitting portion 150 and the second light emitting portion 151) in the light receiving portion 140.

Specifically, a timing at which light based on the first light emitting portion 150 is received by the light receiving portion 140 and a timing at which light based on the second light emitting portion 151 is received by the light receiving portion 140 may be different from each other. Here, when taking into consideration that light is applied from the light emitting portion, the light is reflected by a subject, and reflected light is received by the light receiving portion 140, strictly speaking, a light emission timing is different from a light reception timing. However, a time difference between the light emission timing and the light reception timing is not required to be considered much when taking into consideration speed of light or a small optical path length.

In other words, it is preferable to control light emission timings so that the first light emitting portion 150 and the second light emitting portion 151 exclusively emit light. Light reception in the light receiving portion 140 may be performed at a predetermined timing (for example, a timing corresponding to a predetermined clock signal), but it is necessary to clearly differentiate a process on the first detection signal from a process on the second detection signal in a detection analog circuit. As an example, two circuits such as a first analog circuit for the first detection signal and a second analog circuit for the second detection signal may be prepared, the first analog circuit may be operated at a light emission timing in the first light emitting portion 150, and the second analog circuit may be operated at a light emission timing in the second light emitting portion 151.

Since the first light emitting portion 150 and the second light emitting portion 151 exclusively emit light, the first detection signal and the second detection signal can be processed separately from each other. However, as described above, in order to perform an appropriate body movement noise reduction process, it is important for the first detection signal and the second detection signal to have a certain extent of correlation. In other words, in a case where a noise reduction process is performed on the first detection signal, it is not preferable that the second detection signal to be used is a signal acquired at a timing which is temporally greatly different from a timing of the first detection signal. This is because, if an acquisition timing (light reception timing) of the first detection signal is greatly different from an acquisition timing of the second detection signal, a user's state may change, and thus correlation between the two signals may be very low.

Thus, it is preferable to change a state of acquiring of the first detection signal and a state of acquiring the second detection signal at a high frequency. For example, in a case where a second timing is later than a first timing, a third timing is later than the second timing, and a fourth timing is later than the third timing, the processing unit 200 may acquire the first detection signal in the light receiving portion 140 at the first timing and the third timing, and may acquire the second detection signal in the light receiving portion at the second timing and the fourth timing.

In the above-described way, an acquired detection signal changes from the first detection signal to the second detection signal between at least the first timing and the subsequent second timing (including the second timing). Similarly, characteristics of a detection signal also changes between the second timing and the subsequent third timing, and between the third timing and the subsequent fourth timing. Therefore, for example, in a case where a process is performed on the second detection signal at the second timing and the first detection signal at a predetermined timing as a set, the first detection signal is acquired at least at the first timing and the third timing, and thus it is possible to prevent a time difference between the two detection signals from considerably increasing, that is, to prevent a correlation between the two signals from being considerably lowered.

From the viewpoint of reducing a difference between acquisition timings of two detection signals, the second detection signal used for a noise reduction process on the first detection signal may be acquired at a timing close to a timing of the first detection signal. Specifically, in a case where the second timing is a timing next to the first timing, the third timing is a timing next to the second timing, and the fourth timing is a timing next to the third timing, the processing unit 200 may acquire the first detection signal in the light receiving portion 140 at the first timing and the third timing, and may acquire the second detection signal in the light receiving portion 140 at the second timing and the fourth timing. The light emission intensity of the light emitting portion may be changed at adjacent timings such as the first timing and the second timing, the second timing and the third timing, or the third timing and the fourth timing. In this case, if the light emission intensity is increased at a timing corresponding to the first detection signal, an SN ratio of the first detection signal is improved, and thus it is possible to calculate an appropriate pulse rate. If the light emission intensity is increased at a timing corresponding to the second detection signal, an SN ratio of the second detection signal is improved, and thus it is possible to perform noise reduction with higher accuracy in a noise reduction process.

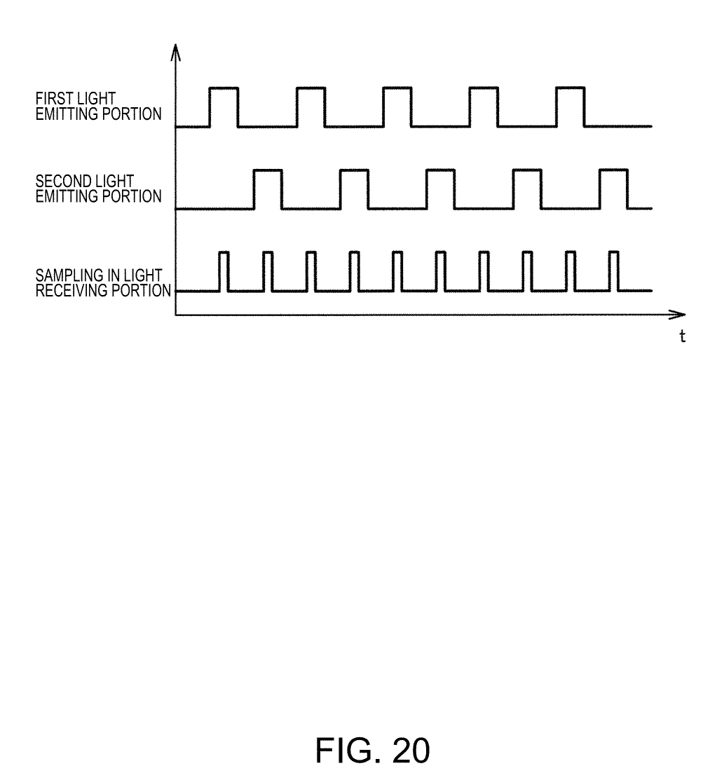

A description has been made of acquisition timings of the first detection signal and the second detection signal at four adjacent timings here, but the first detection signal and the second detection signal may be alternately acquired at other timings. FIG. 20 illustrates a control example in this case. In FIG. 20, a transverse axis expresses time. In control illustrated in FIG. 20, the first light emitting portion 150 and the second light emitting portion 151 alternately emit light in synchronization with light reception timings in the light receiving portion 140. Control may be performed so that the amplitude of a control signal for the first light emitting portion is different from the amplitude of a control signal for the second light emitting portion.

The above timing control is based on a case where a single light receiving portion 140 is shared by a plurality of light emitting portions. In contrast, in a configuration in which a single light receiving portion receives light from a single light emitting portion as in a case where a plurality of light receiving portions are included in the sensor unit 40, each of the plurality of light emitting portions may not exclusively emit light. For example, the processing unit 200 may perform a biological information detection process on the basis of the first detection signal in a light receiving portion at the first timing and the second detection signal in a light receiving portion (a second light receiving portion in a narrow sense) at the first timing.

4. Noise Reduction Process

As described above, the processing unit 200 of the biological information detection device according to the present embodiment performs a correction process on the first detection signal on the basis of the second detection signal, and performs a biological information detection process on the basis of the corrected first detection signal. The processing unit 200 performs a body movement noise reduction process of reducing body movement noise included in the detection signal during the correction process. Consequently, it is possible to prevent the influence of body movement noise and thus to obtain biological information with high accuracy.

Hereinafter, the body movement noise reduction process performed by the processing unit 200 will be described. Specifically, a description will be made of a spectrum subtraction method performed on the basis of the second detection signal, and adaptive filter processing performed on the basis of a signal from a motion sensor.

4.1 Spectrum Subtraction Method

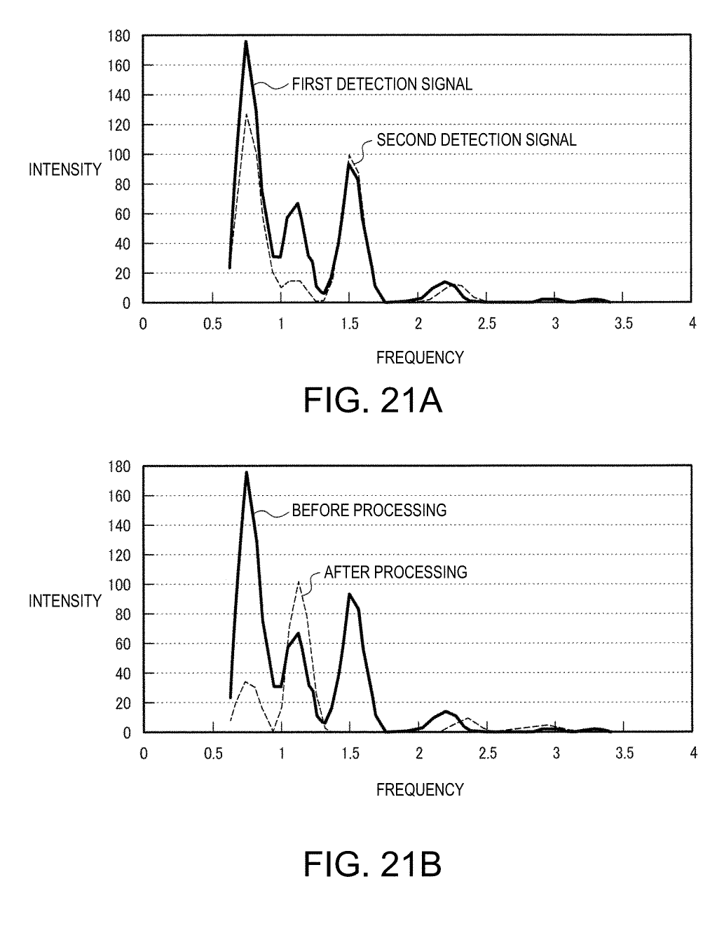

FIGS. 21(A) and 21(B) are diagrams for explaining a noise reduction process on the first detection signal, performed on the basis of the second detection signal according to the spectrum subtraction method. In the spectrum subtraction method, a frequency conversion process is performed on each of the first and second detection signals, and thus a spectrum thereof is obtained. A noise spectrum is estimated from the spectrum of the second detection signal, and a process of subtracting the estimated noise spectrum from the spectrum of the first detection signal is performed.

FIG. 21(A) illustrates a spectrum of the first detection signal and a spectrum of the second detection signal, actually obtained. As described above, as a result of using the biological information detection device 400 according to the present embodiment, the spectrum of the second detection signal is mainly a spectrum corresponding to a noise component. In other words, it can be estimated that a frequency at which a large peak in the spectrum of the second detection signal is a frequency corresponding to body movement noise. Actually, only a peak may be subtracted from the spectrum of the second detection signal, but this is only an example, and, for example, a process of subtracting the entire spectrum of the second detection signal from the entire spectrum of the first detection signal may be performed.

In the subtraction, for example, a coefficient is multiplied by one of the first detection signal and the second detection signal so that noise is canceled out. This coefficient is obtained on the basis of the signal intensity at a predetermined frequency. Alternatively, the coefficient maybe calculated so that noise and a signal are separated from each other according to, for example, a clustering method, and noise of the first detection signal and noise of the second detection signal have the same level.

FIG. 21(B) illustrates an example of the first detection signal before and after the body movement noise reduction process using the spectrum subtraction method. As can be seen from FIG. 21(B), through the body movement noise reduction process, body movement noise appearing at 0.7 to 0.8 Hz (42 to 48 in terms of a pulse rate) and 1.5 Hz (90 in terms of a pulse rate) is reduced, and thus it is possible to reduce a probability that this noise may be wrongly determined as a pulse signal. On the other hand, a signal level can be maintained without being reduced with respect to a spectrum corresponding to a pulse signal appearing at about 1.1 Hz (66 in terms of a pulse rate).

Since the spectrum subtraction method is performed by using a frequency conversion process such as fast Fourier transform (FFT) and a subtraction process in a spectrum, there is an advantage in that an algorithm is simple and a computation amount is small. The spectrum subtraction method has a feature that instantaneous responsiveness is high since there is no learning element as in an adaptive filter processing which will be described later.

4.2 Adaptive Filter Processing

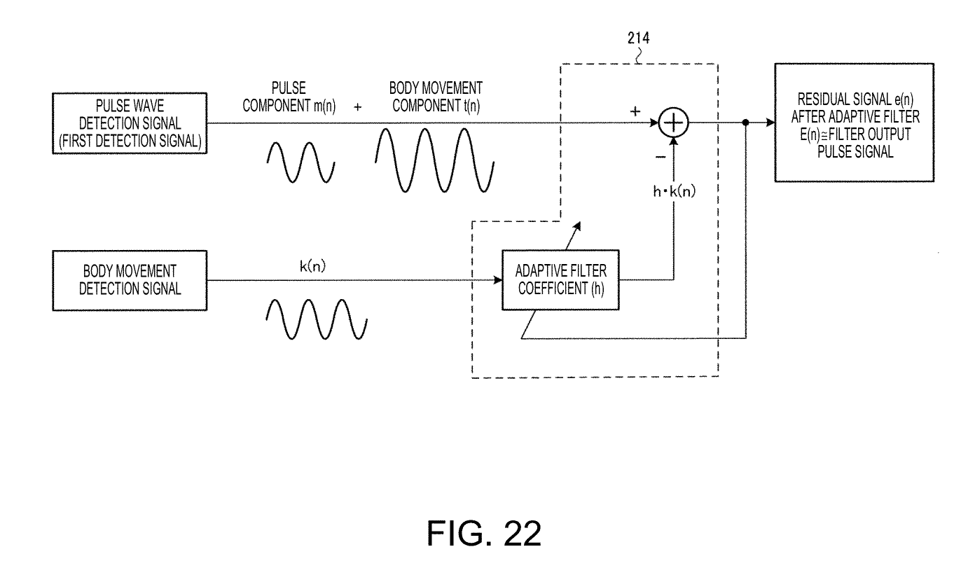

Next, a description will be made of a body movement noise reduction process (second body movement noise reduction process) performed on the basis of a detection signal from a motion sensor by using adaptive filter processing. FIG. 22 illustrates a specific example of a noise reduction process using an adaptive filter. Specifically, since a detection signal in the motion sensor corresponds to body movement noise, a process of subtracting a noise component specified on the basis of the detection signal from the first detection signal is performed, and thus the general concept is the same as in the spectrum subtraction method.

However, even if body movement noise in a pulse wave detection signal and a body movement detection signal from a body movement sensor are all caused by the same body movement, signal levels thereof cannot be said to be the same as each other. Therefore, an estimated body movement noise component is calculated by performing filter processing of adaptively determining a filter coefficient with respect to the body movement detection signal, and a difference between the pulse wave detection signal and the estimated body movement noise component is obtained. Since the filter coefficient is determined adaptively (through learning), the accuracy of the noise reduction process can be improved, but it is necessary to take into consideration a processing load in determination of a filter coefficient or output delay. The adaptive filter processing is a well-known method, and thus a detailed description thereof will be omitted.

In the present embodiment, as illustrated in FIG. 5, the biological information detection device includes the motion sensor (acceleration sensor 172), and the processing unit 200 performs the second body movement noise reduction process of reducing body movement noise of the first detection signal on the basis of a detection signal from the motion sensor.

In other words, the present embodiment is based on a case where the body movement noise reduction process using the second detection signal is performed, but a body movement noise reduction process using the motion sensor may also be performed simultaneously. In the above-described way, it is possible to reduce body movement noise with higher accuracy than in a case where only a body movement noise reduction process using the second detection signal is performed. For example, in FIG. 21(B), overall noise at 0.7 to 0.8 Hz or at 2.3 to 2.4 Hz cannot be reduced, but such noise can be reduced by performing a process using a detection signal from the motion sensor simultaneously.

The processing unit 200 may perform the body movement noise reduction process on the first detection signal on the basis of the second detection signal, and perform the second body movement noise reduction process on a signal having undergone the body movement noise reduction process on the basis of a detection signal from the motion sensor.

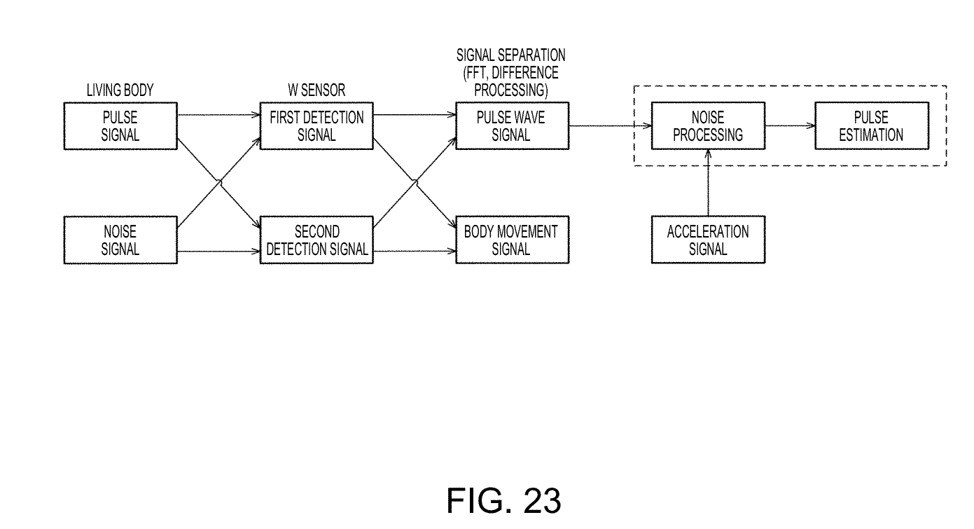

Consequently, it is possible to perform a plurality of body movement noise reduction processes in a predetermined order. Herein, as illustrated in the functional block diagram of FIG. 5, first, the body movement noise reduction process using the second detection signal is performed, and, then, the second body movement noise reduction process is performed. FIG. 23 illustrates a flow of each signal in this case.

As illustrated in FIG. 23, a pulse signal and a noise signal can be detected from a living body, and both of the two signals are included in each of a plurality of detection signals. However, in the present embodiment, a ratio therebetween differs for each detection signal, and a ratio of a pulse signal is high in the first detection signal, and a ratio of a pulse signal in the second detection signal is lower than in the first detection signal (a ratio of body movement noise is high). A pulse signal and a body movement signal (body movement noise) are separated from each other by using the two detection signals. This process is performed according to the above-described spectrum subtraction method. The second body movement noise reduction process using a detection signal (an acceleration signal in FIG. 23) from the motion sensor is performed on the separated pulse signal (the first detection signal having undergone the body movement noise reduction process), and a pulse rate or the like is estimated on the basis of a result thereof.

In the above description, the first detection signal which mainly includes a pulse wave component is acquired under the first pressure P1 as high pressure, and the second detection signal which mainly includes body movement noise is acquired under the second pressure P2 as relatively low pressure. However, a method of the present embodiment is not limited thereto. As described above with reference to FIG. 8, even in a case where pressure is excessively high, a pulse wave component is reduced, and thus a ratio of a body movement noise component relatively increases. In other words, instead of a configuration in which a component corresponding to relatively high pressure is a pulse wave component, and a component corresponding to relatively low pressure is a body movement noise component, there may be the occurrence of a modification in which a component corresponding to relatively high pressure is a body movement noise component, and a component corresponding to relatively low pressure is a pulse wave component. For example, in the example illustrated in FIG. 8, the first pressure P1 as high pressure may employ P1>p4, and the second pressure P2 as low pressure may employ p3<P2<p4 or p2<P2<p3.

In this case, the processing unit 200 performs a correction process on the second detection signal on the basis of the first detection signal, and performs a biological information detection process on the basis of the corrected second detection signal.

Although the present embodiment has been described in detail, it is easily understood by a person skilled in the art that various modifications may occur without substantially departing from the novel matters and effects of the invention. Therefore, such modification examples are all intended to be included in the scope of the invention. For example, in the specification or the drawings, a terminology which is described at least once along with another terminology which has a broader meaning or the same meaning may be replaced with another terminology in any location of the specification or the drawings. Configurations and operations of the biological information detection device and the like are not limited to those described in the present embodiment and may be variously modified.

### REFERENCE SIGNS LIST

Sf SKIN SURFACE, WI WIRE, 10 BAND, 12 BAND HOLE,
14 BUCKLE, 15 BAND INSERTION PORTION, 16 PROTRUSION,
30 CASE,
32 LIGHT EMISSION WINDOW, 34 TOP CASE, 35 TERMINAL PORTION,
36 BOTTOM CASE,
40 SENSOR UNIT, 50 LIGHT TRANSMISSIVE MEMBER,
52 PROTRUSION, 60 RESIN,
70 LIGHT BLOCKING MEMBER, 140 LIGHT RECEIVING PORTION,
150 FIRST LIGHT EMITTING PORTION
151 SECOND LIGHT EMITTING PORTION, 160 BOARD,
161 SECOND BOARD,
162 HEIGHT ADJUSTMENT MEMBER, 170 MOTION SENSOR UNIT,
172 ACCELERATION SENSOR, 175 VIBRATION GENERATION UNIT,
180 FRAME PORTION,
181 FIRST MEMBER, 182 SECOND MEMBER, 200 PROCESSING UNIT,
210 SIGNAL PROCESSOR,
212 BODY MOVEMENT NOISE REDUCING PORTION,
214 SECOND BODY MOVEMENT NOISE REDUCING PORTION,
220 BEATING INFORMATION CALCULATION PORTION,
230 NOTIFICATION CONTROL PORTION, 240 STORAGE UNIT,
250 COMMUNICATION UNIT, 252 ANTENNA,
260 NOTIFICATION UNIT,
400 BIOLOGICAL INFORMATION DETECTION DEVICE, 410 WRIST,
420 TERMINAL APPARATUS, 430 DISPLAY UNIT

1. A sensor unit comprising:
a first light emitting portion that emits light toward a subject;
a second light emitting portion that emits light toward the subject; and
a light receiving portion that receives light from the subject,
wherein, in a case where a height of a contact position or a contact region with the subject in a position or a region corresponding to the first light emitting portion is indicated by H1, and a height of a contact position or a contact region with the subject in a position or a region corresponding to the second light emitting portion is indicated by H2, a relationship of H1>H2 is satisfied.

2. The sensor unit according to claim 1,
wherein H1 indicates a height of a contact position or a contact region with the subject in an arrangement region of the first light emitting portion, and
wherein H2 indicates a height of a contact position or a contact region with the subject in an arrangement region of the second light emitting portion.

3. The sensor unit according to claim 2,
wherein, in a case where a height to a surface of the first light emitting portion on the subject side is indicated by HA1, and a height to a surface of the second light emitting portion on the subject side is indicated by HA2, a relationship of HA1>HA2 is satisfied, and thus the relationship of H1>H2 is satisfied.

4. The sensor unit according to claim 3,
wherein the second light emitting portion and the light receiving portion are provided on a board, and
wherein a height adjustment member is provided between the first light emitting portion and the board.

5. The sensor unit according to claim 4,
wherein the height adjustment member is a second board, and
wherein an external connection terminal of the first light emitting portion is connected to a connection terminal provided on the board via a through hole of the second board.

6. The sensor unit according to claim 4,
wherein an external connection terminal of the first light emitting portion is connected to a connection terminal provided on the board via a wire.

7. The sensor unit according to claim 2,
wherein, in a case where a direction directed toward the subject from the sensor unit is set to a first direction when biological information is detected, a length of the first light emitting portion in the first direction is indicated by LH1, and a length of the second light emitting portion in the first direction is indicated by LH2, a relationship of LH1>LH2 is satisfied, and thus the relationship of H1>H2 is satisfied.

8. The sensor unit according to claim 1, further comprising:
a first member provided between at least the first light emitting portion and the light receiving portion; and
a second member provided between at least the second light emitting portion and the light receiving portion,
wherein, in a case where a height of the first member is indicated by HC1, and a height of the second member is indicated by HC2, a relationship of HC1>HC2 is satisfied, and thus the relationship of H1>H2 is satisfied.

9. The sensor unit according to claim 1, further comprising:
a light transmissive member that is provided at a position located further toward the subject side than the first light emitting portion, transmits light from the subject therethrough, and comes into contact with the subject so as to apply pressure thereto when biological information of the subject is measured,
wherein, in a case where a height of the light transmissive member in a position or a region corresponding to the first light emitting portion is indicated by HD1, a relationship of HD1>H2 is satisfied, and thus the relationship of H1>H2 is satisfied.

10. The sensor unit according to claim 9,
wherein the light transmissive member is provided at a position located further toward the subject side than the second light emitting portion, transmits light from the subject therethrough, and comes into contact with the subject so as to apply pressure thereto when biological information of the subject is measured,
wherein, in a case where a height of the light transmissive member in a position or a region corresponding to the second light emitting portion is indicated by HD2, a relationship of HD1>HD2 is satisfied, and thus the relationship of H1>H2 is satisfied.

11. The sensor unit according to claim 1,
wherein, in a case where a height of a contact position or a contact region with the subject in a position or a region corresponding to the light receiving portion is indicated by H3, a relationship of H1≧H3≧H2 (here, a case of H1=H3=H2 is excluded) is satisfied.

12. The sensor unit according to claim 1,
wherein the light receiving portion receives light from the subject in a case where pressure applied to a measurement part of the subject is first pressure, and light from the subject in a case where the pressure applied to the measurement part of the subject is second pressure lower than the first pressure.

13. The sensor unit according to claim 1,
wherein a height of the contact region with the subject is an average height of heights at respective points included in the contact region.

14. A biological information detection device comprising the sensor unit according to claim 1.

15. The biological information detection device according to claim 14, further comprising:
a processing unit that performs a biological information detection process on the basis of a first light reception result which is a light reception result of light from the subject, corresponding to light from the first light emitting portion, and a second light reception result which is a light reception result of light from the subject, corresponding to light from the second light emitting portion.

16. A biological information detection device comprising:
at least one light emitting portion that emits light toward a subject;
at least one light receiving portion that receives light from the subject; and
a processing unit that performs a biological information detection process on the basis of a detection signal output from the light receiving portion,
wherein the processing unit performs the biological information detection process on the basis of a first detection signal which is the detection signal in a case where pressure applied to a measurement part of the subject is first pressure, and a second detection signal which is the detection signal in a case where the pressure applied to the measurement part of the subject is second pressure lower than the first pressure.

17. The biological information detection device according to claim 16,
wherein the processing unit performs the biological information detection process on the basis of the first detection signal in the light receiving portion at a first timing, and the second detection signal in the light receiving portion at a second timing which is different from the first timing.

18. The biological information detection device according to claim 17, further comprising:
a first light emitting portion and a second light emitting portion as the light emitting portion,
wherein the processing unit performs the biological information detection process on the basis of the first detection signal in the light receiving portion at the first timing, based on light emission of the first light emitting portion, and the second detection signal in the light receiving portion at the second timing, based on light emission of the second light emitting portion.

19. The biological information detection device according to claim 17,
wherein, in a case where the second timing is a timing subsequent to the first timing, a third timing is a timing subsequent to the second timing, and a fourth timing is a timing subsequent to the third timing, the processing unit acquires the first detection signal in the light receiving portion at the first timing and the third timing, and acquires the second detection signal in the light receiving portion at the second timing and the fourth timing.

20. The biological information detection device according to claim 16,
wherein the processing unit performs the biological information detection process on the basis of the first detection signal in the light receiving portion at the first timing, and the second detection signal in the light receiving portion at the first timing.

21. The biological information detection device according to claim 16,
wherein the processing unit performs a correction process on the first detection signal on the basis of the second detection signal, and performs the biological information detection process on the basis of the corrected first detection signal.

22. The biological information detection device according to claim 16,
wherein the processing unit performs a correction process on the second detection signal on the basis of the first detection signal, and performs the biological information detection process on the basis of the corrected second detection signal.

23. The biological information detection device according to claim 21,
wherein the processing unit performs a body movement noise reduction process of reducing body movement noise included in the detection signal as the correction process.

24. An electronic apparatus comprising the sensor unit according to claim 1.

25. An electronic apparatus comprising the biological information detection device according to claim 14.

26. A biological information detection method for a biological information detection device including at least one light emitting portion that emits light toward a subject, and at least one light receiving portion that receives light from the subject, the method comprising:
performing the biological information detection process on the basis of a first detection signal which is the detection signal in a case where pressure applied to a measurement part of the subject is first pressure, and a second detection signal which is the detection signal in a case where the pressure applied to the measurement part of the subject is second pressure lower than the first pressure.
